(19) United States
(12) Patent Application Publication
(10) Pub. No.: US 2022/0150284 A1
(43) Pub. Date: May 12, 2022

(54) RESOURCE MANAGEMENT SYSTEM, RESOURCE MANAGEMENT APPARATUS, AND RECORDING MEDIUM

(71) Applicant: Ryo SUZUKI, Kanagawa (JP)

(72) Inventor: Ryo SUZUKI, Kanagawa (JP)

(73) Assignee: Ricoh Company, Ltd., Tokyo (JP)

(21) Appl. No.: 17/453,242

(22) Filed: Nov. 2, 2021

(30) Foreign Application Priority Data
Nov. 11, 2020 (JP) 2020-187768

(51) Int. Cl. H04L 29/06; G06Q 10/02; G16H 10/60

(52) U.S. Cl. CPC H04L 65/1073; H04L 65/403; G06F 3/0482; G16H 10/60; G06Q 10/02

(57) ABSTRACT

A resource management system includes a resource management apparatus configured to manage a use state of a resource; and a communication terminal configured to communicate with the resource management apparatus. The communication terminal executes acquiring code information embedded in a predetermined code by capturing the predetermined code relating to reservation information, the predetermined code being displayed by a resource terminal used in a predetermined resource and configured to communicate with the resource management apparatus; and transmitting, to the resource management apparatus, user identification information for identifying a user using the predetermined resource associated with the acquired code information, as a start request to start using the predetermined resource. The resource management apparatus executes receiving the user identification information transmitted by the communication terminal, and registering the user identification information and resource use state information representing the use state of the predetermined resource, based on the received user identification information.

### CROSS-REFERENCE TO RELATED APPLICATION

The present application is based on and claims priority under 35 U.S.C. § 119 to Japanese Patent Application No. 2020-187768, filed on Nov. 11, 2020, the contents of which are incorporated herein by reference in their entirety.

### BACKGROUND OF THE INVENTION

### 1. Field of the Invention

The present invention relates to a resource management system, a resource management apparatus, and a recording medium.

### 2. Description of the Related Art

There is known a technique for displaying information relating to a reservation of a conference room, or the like, on an information processing terminal which is installed or used in the conference room. With this technique, a user can use the conference room by inputting the necessary information into the information processing terminal, such as when starting to use the conference room.

Accordingly, it is disclosed that when reserving a resource such as a conference room, in a case where an administrator sets a setting file in a second conference room terminal 90B, when the administrator captures an image code including connection destination information with the second conference room terminal 90B, an in-terminal server 98 can be accessed (see, for example, Patent Document 1).

Patent Document 1: Japanese Unexamined Patent Application Publication No. 2020-123072

### SUMMARY OF THE INVENTION

According to one aspect of the present invention, there is provided a resource management system including a resource management apparatus configured to manage a use state of a predetermined resource; and a communication terminal configured to communicate with the resource management apparatus, wherein the communication terminal includes first circuitry; and a first memory storing computer-executable instructions that cause the first circuitry to execute acquiring code information embedded in a predetermined code by capturing the predetermined code relating to predetermined reservation information of the predetermined resource, the predetermined code being displayed by a resource terminal that is used in the predetermined resource and configured to communicate with the resource management apparatus; and transmitting, to the resource management apparatus, user identification information for identifying a user using the predetermined resource associated with the code information acquired at the acquiring, as a start request to start using the predetermined resource, and wherein the resource management apparatus includes second circuitry; and a second memory storing computer-executable instructions that cause the second circuitry to execute receiving the user identification information transmitted by the communication terminal, and registering the user identification information and resource use state information representing the use state of the predetermined resource, based on the user identification information received at the receiving.

### BRIEF DESCRIPTION OF THE DRAWINGS

FIG. 1 is a diagram illustrating an example of the overall configuration of a communication system according to a first embodiment of the present invention;

FIG. 2 is a diagram illustrating an example of a hardware configuration of a resource management apparatus and a reservation management apparatus according to the first embodiment of the present invention;

FIG. 3 is a diagram illustrating an example of a hardware configuration of a user terminal and a resource terminal according to the first embodiment of the present invention;

FIG. 4 is a diagram illustrating an example of a functional configuration of a communication system according to the first embodiment of the present invention;

FIG. 5A is a conceptual diagram illustrating an example of a login management table according to the first embodiment of the present invention;

FIG. 5B is a conceptual diagram illustrating an example of an infected person management table according to the first embodiment of the present invention;

FIG. 6 is a conceptual diagram illustrating an example of a resource state information table according to the first embodiment of the present invention;

FIG. 7 is a conceptual diagram illustrating an example of a reservation information management table according to the first embodiment of the present invention;

FIG. 8 is a sequence diagram illustrating an example of an authentication process according to the first embodiment of the present invention;

FIG. 9 is a sequence diagram illustrating an example of a check in process using a QR code according to the first embodiment of the present invention;

FIG. 10 is a sequence diagram illustrating an example of a check in process using a QR code according to the first embodiment of the present invention;

FIG. 11 is a sequence diagram illustrating an example of a registration process of an infected person according to the first embodiment of the present invention;

FIG. 12 is a sequence diagram illustrating an example of a contact person reporting process according to the first embodiment of the present invention;

FIG. 13 13 is a diagram illustrating an example of an authentication screen according to the first embodiment of the present invention;

FIG. 14 is a diagram illustrating an example of a check in possible screen on a resource terminal according to the first embodiment of the present invention;

FIG. 15 is a diagram illustrating an example of a QR code capturing screen according to the first embodiment of the present invention;

FIG. 16 is a diagram illustrating an example of an expected participant list display screen according to the first embodiment of the present invention;

FIG. 17 is a diagram illustrating an example of an infected person registration screen according to the first embodiment of the present invention;

FIG. 18 is a diagram illustrating an example of a contact confirmation screen according to the first embodiment of the present invention;

FIG. 19 is a diagram illustrating another example of the contact confirmation screen according to the first embodiment of the present invention;

FIG. 20 is a sequence diagram illustrating an example of a process for determining a contact person and a process for reporting to a contact person according to a second embodiment of the present invention;

FIG. 21 is a flowchart illustrating an example of a process for determining a contact person according to the second embodiment of the present invention; and

FIG. 22 is a diagram illustrating an example of an alert screen according to the second embodiment of the present invention.

### DESCRIPTION OF THE EMBODIMENTS

With the conventional technology, it is possible to manage a user, such as an administrator or a participant, who plans to use a resource such as a conference room, but there is a problem that it is difficult to identify the user who has actually used the resource.

Hereinafter, embodiments for carrying out the invention will be described with reference to the drawings. In the description of the drawings, the same elements are denoted by the same reference numerals, and overlapping descriptions are omitted.

### First Embodiment

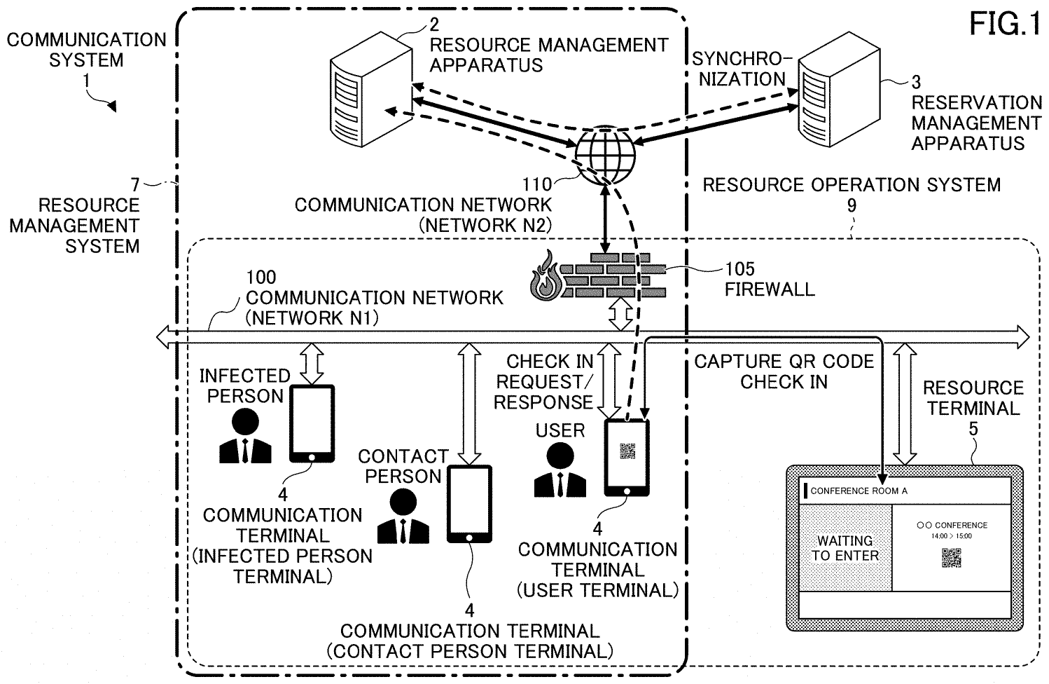

First, the first embodiment will be described with reference to FIGS. 1 to 19.

### [Overview of Communication System]

### <Example of System Configuration>

FIG. 1 is a diagram illustrating an example of the overall configuration of a communication system according to the first embodiment. According to the present embodiment, a communication system 1 includes a resource management apparatus 2, a reservation management apparatus 3, a communication terminal 4, and a resource terminal 5. The communication terminal 4 and the resource terminal 5 are connected to each other via a communication network 100, such as an in-house network (a network N1; hereinafter, simply referred to as the communication network 100), inside a firewall 105. The resource management apparatus 2 and the reservation management apparatus 3 are connected to each other via a communication network 110 (a network N2; hereinafter, simply referred to as the communication network 110) outside the firewall 105. The communication network 110 is a network in which an unspecified number of communications is transmitted and is constructed by the Internet, a mobile communication network, a local area network (LAN), or the like. That is, the resource management apparatus 2 is capable of communicating with the reservation management apparatus 3 via the communication network 110. The resource management apparatus 2 is capable of communicating with the communication terminal 4 and the resource terminal 5 via the communication network 110, the firewall 105, and the communication network 100. The reservation management apparatus 3 is capable of communicating with the communication terminal 4 and the resource terminal 5 via the communication network 110, the firewall 105, and the communication network 100. The communication terminal 4 is capable of communicating with the resource terminal 5 via the communication network 100.

The communication network 110 may include, not only wired communication networks, but also wireless communication networks, such as 3rd Generation (3G), 4th Generation (4G), 5th Generation (5G), Worldwide Interoperability for Microwave Access (WiMAX), Long Term Evolution (LTE), or the like.

### <<Resource Management Apparatus>>

The resource management apparatus 2 is implemented by one or more information processing apparatuses (computer systems) in which a typical server operating system (OS) or the like is installed. The resource management apparatus 2 acquires the reservation information of each resource and the expected participant identification information set in the reservation information from the reservation management apparatus 3, as a process relating to the communication network 110. This expected participant identification information is information for identifying an expected participant who is scheduled to participate in the reserved resource, and, for example, an e-mail address is used as the expected participant identification information. Further, as a process relating to the communication network 100, the check in and the check out with respect to each conference room are managed, and communication with the resource terminal 5 installed in each conference room is performed based on the reservation information of each conference room acquired from the reservation management apparatus 3. In the present embodiment, the resource management apparatus 2 functions as an example of a resource management apparatus.

The communication network 100 includes, for example, conference rooms within a floor space, workplaces of expected participants, or the like. The expected participants who are scheduled to participate in an event held in a predetermined conference room, can access the communication network 110 from each location via the communication network 100 and the firewall 105 from each location. The communication network 100 is, for example, a local area network (LAN). However, it is not necessary that each conference room and the workplaces of the expected participants are within the same in-house LAN.

Further, the information shared by the resource management apparatus 2 is not limited to text information, and may be information such as images, movies, sound, or the like. However, for the sake of convenience, it is described in the present embodiment that text information is mainly used for notifications (reports).

Similarly, the resource management apparatus 2 can manage a user with an account different from the account of the reservation management apparatus 3, but the resource management apparatus 2 described above does not need to be aware of the account of the resource management apparatus 2 in principle. However, the resource management apparatus 2 can specify and notify any account of the resource management apparatus 2.

### <<Reservation Management Apparatus>>

The reservation management apparatus 3 is implemented by one or more information processing apparatuses (computer systems) in which a typical server OS or the like is installed. A system relating to the reservation of resources and the reservation of a user or the management of a schedule, provided by the reservation management apparatus 3 is referred to herein as a reservation management system. The reservation management apparatus 3 includes a calendar system and provides a web application for managing various scheduled events of a user. For example, the reservation management apparatus 3 includes the following functions.

To accept the registration of a reservation or a schedule of the user and allow the user to confirm his or her reservation status or schedule at any time from anywhere.

To send a report to the user by e-mail at a set date and time, such as 30 minutes prior to the scheduled event.

To enable one person to manage the reservation or the schedule by multiple calendars (for example, for work, for home, or the like)

To share the calendar with people in the same group. The reservation management apparatus 3 manages a user by an account. An account is a user's right to use a service. In many systems, the user logs into the system by using an account. For this reason, the account has a function of uniquely identifying the user by the system (identification information function). According to the present embodiment, the reservation management apparatus 3 transmits the reservation information of a previously registered account (domain) to the resource management apparatus 2 that manages conference rooms or the like. Alternatively, by specifying the account of the reservation management apparatus 3 and requesting the reservation information by the resource management apparatus 2, the resource management apparatus 2 may acquire the reservation information of the conference room from the reservation management apparatus 3. The account is not particularly limited as long as information by which a user can be uniquely identified is used, e.g., an e-mail address, an ID, a phone number, or the like.

In the present embodiment, the reservation management apparatus 3 can manage reservation information representing the reservations of various resources, including free spaces and conference rooms, and can manage user schedules. As the reservation management system, G Suite (registered trademark), Office 365 (registered trademark), or the like are known, but the reservation management system having the function described in the present embodiment is sufficient.

Further, in the present embodiment, information relating to various resources reserved by each user and the schedule of each user is pre-registered in the reservation management apparatus 3. That is, the web application of the reservation management apparatus 3 is customized according to the reservation person's account, the resource ID, the resource name, the start date and time, the end date and time, the expected participant identification information, or the like in an in-house network of a company using the communication system 1. Accordingly, the reservation information is associated with a resource that includes each conference room of the company using the communication system 1. In the present embodiment, the reservation management apparatus 3 functions as an example of the reservation management apparatus. Further, in the present embodiment, the system including the resource management apparatus 2 and the communication terminal 4 corresponds to a resource management system 7.

### <<Communication Terminal>>

The communication terminal 4 is implemented by one or more information processing apparatuses (computer systems) in which a typical server OS or the like is installed. The communication terminal 4 is for making various settings when a user uses a resource (a conference room) and after using a resource, via a web page provided by the resource management apparatus 2. The communication terminal 4 is capable of communicating in a wired or wireless manner. The communication terminal 4 is used, for example, as a display terminal for displaying the name of the user when starting to use a resource, such as a conference room or the like reserved by the user. Further, the communication terminal 4 may have a function for communicating using browser software and displaying a web page.

The communication terminal 4 may be, for example, a communication terminal having a communication function such as a smartphone, a tablet terminal, a Personal Digital Assistant (PDA), a wearable personal computer (PC) (a sunglasses type, a wristwatch type, or the like). That is, a terminal capable of of operating software such as browser software is used as a communication terminal. In the present embodiment, the communication terminal 4 functions as an example of the communication terminal. In the present embodiment, the communication terminal 4 includes a communication terminal possessed by each user including a reservation person who has reserved a resource described below, users who have used the same resource within a predetermined time when a predetermined resource is used, an infected person who has been found to be infected by a particular infectious disease or who has tested positive for a particular infectious disease, and a contact person who has contacted an infected person.

### <<Resource Terminal>>

The resource terminal 5 is a communication terminal in which a typical OS or the like is installed. The resource terminal 5 may be replaced by a communication terminal having communication functions such as a tablet terminal, a Personal Computer (PC), a Personal Digital Assistant (PDA), a wearable PC (sunglass type, wristwatch type, or the like). That is, a terminal capable of operating software such as browser software is used as a communication terminal.

As will be described later, the resource terminal 5 has a function of generating and displaying, for example, a bar code as an example of one-dimensional code or a QR code (registered trademark; hereinafter, omitted) as an example of two-dimensional code based on the reservation identification information relating to a resource in which the own resource terminal 5 is disposed and installed, and having the communication terminal 4 used by a user read the generated code.

In the present embodiment, the resource terminal 5 may include the resource management apparatus 2 for operating exclusive-use application software (hereinafter, a conference application) related to a predetermined event such as a conference held by the communication system 1. Note that the above-described application may be substituted by browser software. In the present embodiment, the resource terminal 5 functions as an example of the resource terminal. Further, in the present embodiment, the system including the communication terminal 4 and the resource terminal 5 corresponds to a resource operation system 9.

The present embodiment includes a configuration in which the above-described code is displayed on the resource terminal 5 disposed in or used by a reserved predetermined resource, and the communication terminal 4 starts using the resource by capturing the code. In this way, an operation involving the user's physical contact with the resource terminal 5 is avoided at the time when the use of the resource starts or ends, and the risk of being infected by a particular infectious disease by contacting the resource terminal 5 is reduced, thereby realizing the safe use of the resource.

### (Terms)

“Resources” refer to resources owned, leased, or managed by schools, companies, groups, organizations, or the like. Resources of schools, companies, groups, organizations, or the like may include, for example, conference rooms, furnishings available in the conference rooms, devices, persons (reception groups), or the like. Further, free spaces with free addresses (hereinafter simply referred to as free spaces), locations of desks, chairs, booths, or the like, company cars, bicycles, parking lots, bicycle parking spaces, remote conference systems, printing services and mechanisms, or the like may be resources. Resources that are difficult to move are referred to as facilities. In the present embodiment, the term conference room is used as an example of resources for the sake of description. Other examples of resources include various facilities such as various electronic devices, locations such as rental offices, rental studios, or the like, rental cycles, rental cars (sharing cars), lodging facilities such as hotels, event venues, rental spaces such as lockers, or the like.

A request to use a resource may be a request to use a resource for which resource identification information (resource ID) is required. For example, there is a start of use or a start of reservation.

Further, the user includes a reservation person who has reserved a resource, such as a free space, a conference room, or the like, participants who participate in the usage of the resource, and an administrator who manages the communication system 1 according to the present embodiment. In other words, if a person who has reserved a predetermined resource uses or participates in the resource, the person who has reserved the resource becomes the user or the participant.

### [Hardware Configuration]

### <Hardware Configuration of Resource Management Apparatus and Reservation Management Apparatus>

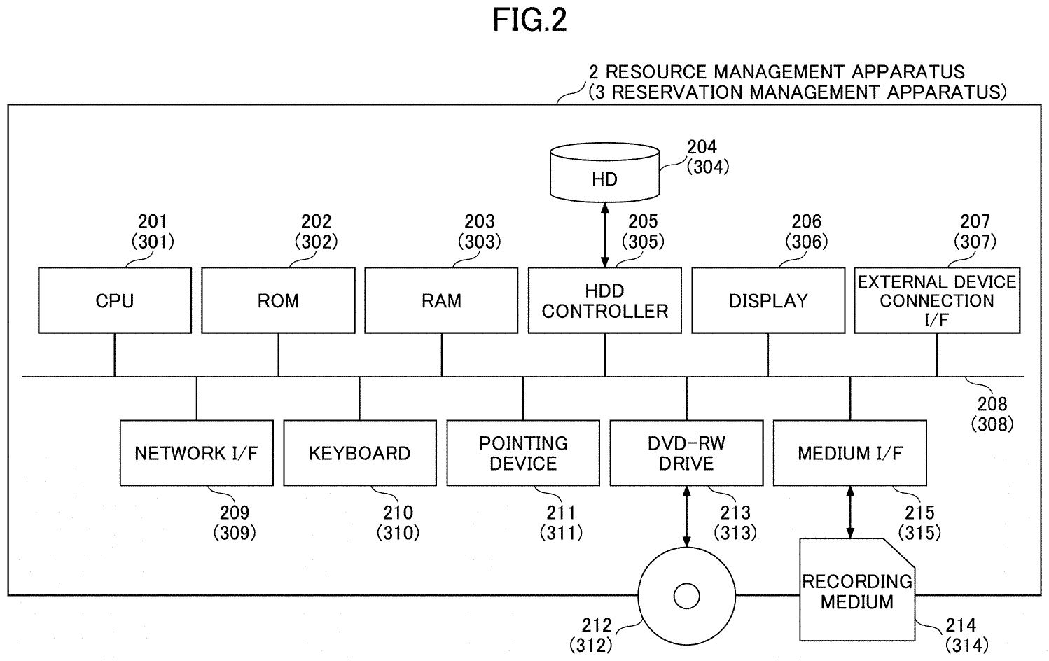

FIG. 2 is a diagram illustrating an example of a hardware configuration of the resource management apparatus and the reservation management apparatus according to the first embodiment. The resource management apparatus 2 and the reservation management apparatus 3 are implemented, for example, by a computer system having a hardware configuration illustrated in FIG. 2.

The resource management apparatus 2 illustrated in FIG. 2 is constructed by a computer and includes, for example, a central processing unit (CPU) 201, a read-only memory (ROM) 202, a random access memory (RAM) 203, a hard drive (HD) 204, a hard disk drive (HDD) controller 205, a display 206, an external device connection interface (I/F) 207, a bus line 208, a network I/F 209, a keyboard 210, a pointing device 211, a Digital Versatile Disk Rewritable (DVD-RW) drive 213, and a medium I/F 215.

Among these, the CPU 201 controls the operation of the entire computer system. The ROM 202 stores a program used to drive the CPU 201, such as an initial program loader (IPL). The RAM 203 is used as a work area of the CPU 201. The HD 204 stores various kinds of data such as a program. The HDD controller 205 controls the reading or writing of various kinds of data to the HD 204 according to the control of the CPU 201. The display 206 displays various kinds of information such as cursors, menus, windows, characters, or images. The external device connection I/F 207 is an interface for connecting various external devices. In this case, the external device may be, for example, a Universal Serial Bus (USB) memory or a printer. The network I/F 209 is an interface for performing data communication using the communication network 110. The bus line 208 is an address bus, a data bus, or the like for electrically connecting elements such as the CPU 201 illustrated in FIG. 2.

The keyboard 210 is a type of input means having a plurality of keys for inputting characters, numbers, various indications, or the like. The pointing device 211 is a type of input means for selecting and executing various instructions, selecting a processing target, moving a cursor, or the like. The DVD-RW drive 213 controls the reading or writing of various kinds of data to a DVD-RW 212 as an example of a removable recording medium. The medium is not limited to a DVD-RW, but may be a Digital Versatile Disc Recordable (DVD-R) or the like. The medium I/F 215 controls the reading or writing (storage) of data to a recording medium 214, such as a flash memory.

The reservation management apparatus 3 is constructed by a computer and, as illustrated in FIG.

2, includes a CPU 301, a ROM 302, a RAM 303, a HD 304, an HDD controller 305, a display 306, an external device connection I/F 307, a bus line 308, a network I/F 309, a keyboard 310, a pointing device 311, a DVD-RW drive 313, and a medium I/F 315. These have similar configurations to the CPU 201, the ROM 202, the RAM 203, the HD 204, the HDD controller 205, the display 206, the external device connection I/F 207, the bus line 208, the network I/F 209, the keyboard 210, the pointing device 211, the DVD-RW drive 213, and the medium I/F 215 in the resource management apparatus 2, and therefore, the descriptions thereof will be omitted.

### <Hardware Configuration of Communication Terminal and Resource Terminal>

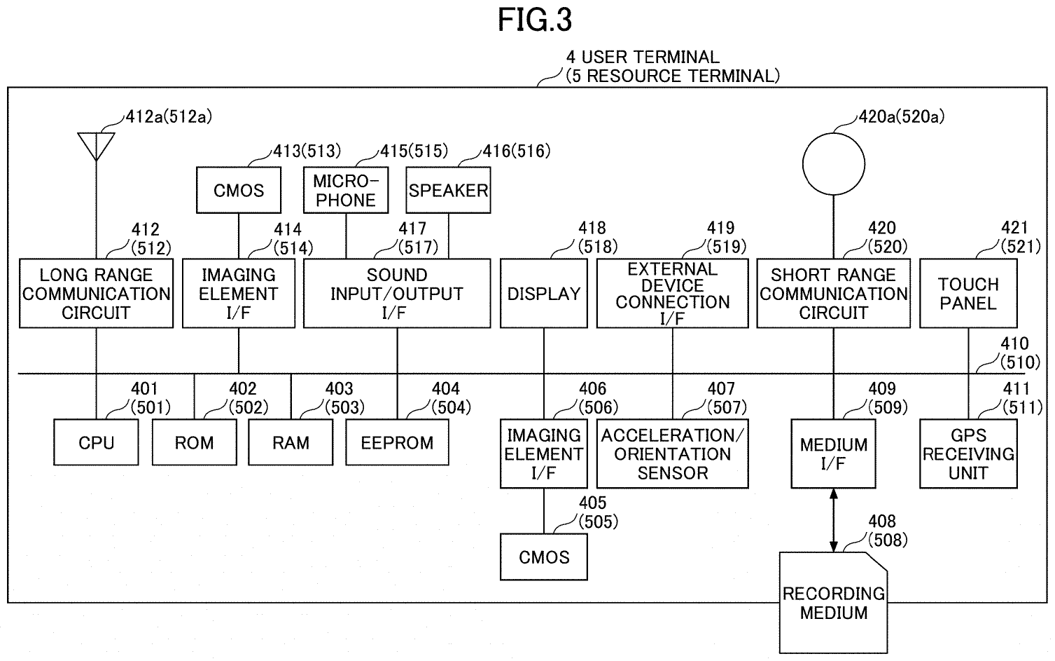

FIG. 3 is a diagram illustrating an example of a hardware configuration of a user terminal and a resource terminal according to the first embodiment. The communication terminal 4 and the resource terminal 5 are implemented, for example, by a computer system having a hardware configuration illustrated in FIG. 3.

The communication terminal 4 illustrated in FIG. 3 is constructed by a computer and includes, for example, a CPU 401, a ROM 402, a RAM 403, an EEPROM 404, a Complementary Metal-Oxide-Semiconductor (CMOS) sensor 405, an imaging element I/F 406, an acceleration/orientation sensor 407, a medium I/F 409, and a global positioning system (GPS) receiving unit 411.

Among these, the CPU 401 controls the entire operation of the resource terminal 5. The ROM 402 stores the CPU 401 and a program used to drive the CPU 401, such as an IPL. The RAM 403 is used as the work area of the CPU 401. The EEPROM 404 reads out or writes various kinds of data, such as applications, according to the control of the CPU 401. The CMOS sensor 405 is a type of built-in imaging means that captures a subject according to the control of the CPU 401 to obtain image data or video data. The imaging means may be an imaging means such as a Charge Coupled Device (CCD) sensor, instead of a CMOS sensor. The imaging element I/F 406 is a circuit that controls the driving of the CMOS sensor 405. The acceleration/orientation sensor 407 includes a variety of sensors, such as an electromagnetic compass which detects geomagnetic fields, a gyrocompass, an acceleration sensor, or the like. The medium I/F 409 controls the reading or writing (storage) of data to a recording medium 408, such as a flash memory. The GPS receiving unit 411 receives the GPS signal from the GPS satellite.

The communication terminal 4 further includes, for example, a long range communication circuit 412, an antenna 412a of the long range communication circuit 412, a CMOS sensor 413, an imaging element I/F 414, a microphone 415, a speaker 416, a sound input/output I/F 417, a display 418, an external device connection I/F 419, a short range communication circuit 420, an antenna 420a of the short range communication circuit 420, and a touch panel 421.

Among these, the long range communication circuit 412 is a circuit for communicating with other devices via the communication network 100. The CMOS sensor 413 is a type of internal imaging means that captures a subject according to the control of the CPU 401 and obtains image data. The imaging element I/F 414 is a circuit that controls the drive of the CMOS sensor 413. The microphone 415 is a built-in circuit that that converts sound to an electrical signal, and acquires sound and sound waves emitted from an external speaker or the like, and acquires information using an electrical signal. The speaker 416 is a built-in circuit that converts electrical signals to physical vibrations to produce sound, such as music and speech. The sound input/output I/F 417 is a circuit that processes the input and output of sound signals between the microphone 415 and the speaker 416 according to the control of the CPU 401.

The display 418 is a type of display means such as a liquid crystal or an organic Electro Luminescence (EL) for displaying an image of a subject or various icons or the like. The external device connection I/F 419 is an interface for connecting various external devices. The short range communication circuit 420 is a communication circuit such as a NFC (Near Field Communication), Bluetooth (registered trademark) or the like. The touch panel 421 is a type of input means for operating the communication terminal 4 by having a user operate the display 418 by a pressing operation, a clicking operation, a tapping operation, or the like. The imaging means, such as the CMOS sensor 413, the short range communication means, such as the short range communication circuit 420, the voice input input means, such as the microphone 415, or the like are information acquiring means for acquiring identification information of a resource by, for example, an image such as an image code, wireless communication, or sound wave, respectively.

The communication terminal 4 includes a bus line 410. The bus line 410 is an address bus, data bus, or the like for electrically connecting elements such as the CPU 401 illustrated in FIG. 3.

The resource terminal 5 is constructed by a computer and includes, as illustrated in FIG. 3, for example, a CPU 501, a ROM 502, a RAM 503, an EEPROM 504, a CMOS sensor 505, an imaging element I/F 506, an acceleration/orientation sensor 507, a medium I/F 509, a bus line 510, and a GPS receiving unit 511.

The resource terminal 5 includes, for example, a long range communication circuit 512, an antenna 512a of the long range communication circuit 512, a CMOS sensor 513, an imaging element I/F 514, a microphone 515, a speaker 516, a sound input/output I/F 517, a display 518, an external device connection I/F 519, a short range communication circuit 520, an antenna 520a of the short range communication circuit 520, and a touch panel 521. These have similar configurations to the CPU 401, the ROM 402, the RAM 403, the EEPROM 404, the CMOS sensor 405, the imaging element I/F406, the acceleration/orientation sensor 407, the medium I/F 409, the bus line 510, the GPS receiving unit 411, the long range communication circuit 412, the antenna 412a of the long range communication circuit 412, the CMOS sensor 413, the imaging element I/F 414, the microphone 415, the speaker 416, the sound input/output I/F 417, the display 418, the external device connection I/F 419, the short range communication circuit 420, the antenna 420a of the short range communication circuit 420, and the touch panel 421 in the communication terminal 4, and thus the descriptions thereof will be omitted.

Each of the above-described programs may be distributed by being recorded as a file in an installable or executable format in a computer-readable recording medium. Examples of a recording medium include a Compact Disc Recordable (CD-R), a Digital Versatile Disk (DVD), a Blu-ray Disc, a secure digital (SD) (registered trademark) card, a USB memory, or the like. The recording medium may also be provided as a program product, either domestically or abroad. For example, the resource management apparatus 2 and the communication terminal 4 implement the resource management method according to an embodiment of the present invention by executing a program according to an embodiment of the present invention.

### [Functional Configuration of Resource Management System]

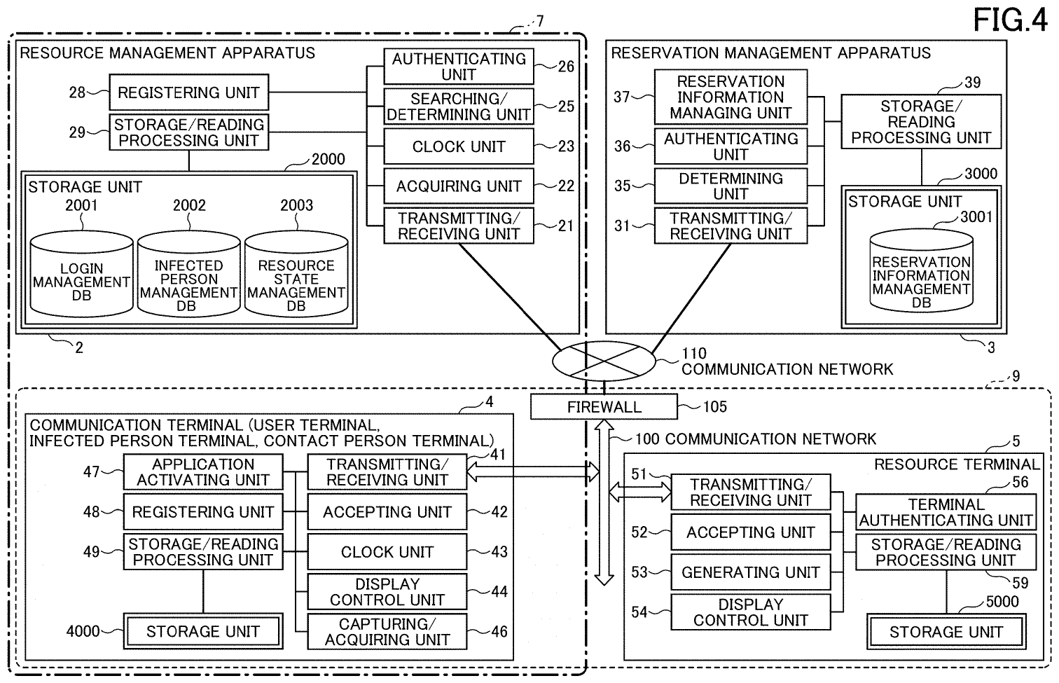

Next, the functional configuration of the present embodiment will be described with reference to FIGS. 4 to 7. FIG. 4 is a diagram illustrating an example of a functional configuration of the communication system according to the first embodiment.

### <Functional Configuration of Resource Management Apparatus>

As illustrated in FIG. 4, the resource management apparatus 2 includes a transmitting/receiving unit 21, an acquiring unit 22, a clock unit 23, a searching/determining unit 25, an authenticating unit 26, a registering unit 28, and a storage/reading processing unit 29. Each of these functional units is a function or means that is implemented by each of the elements illustrated in FIG. 2 operating by an instruction from the CPU 201 according to a program for the resource management apparatus 2 loaded into the RAM 203 from at least one of the ROM 202 or the HD 204. The resource management apparatus 2 includes a storage unit 2000 constructed by the ROM 202 or the HD 204 illustrated in FIG. 2. (Login management table)

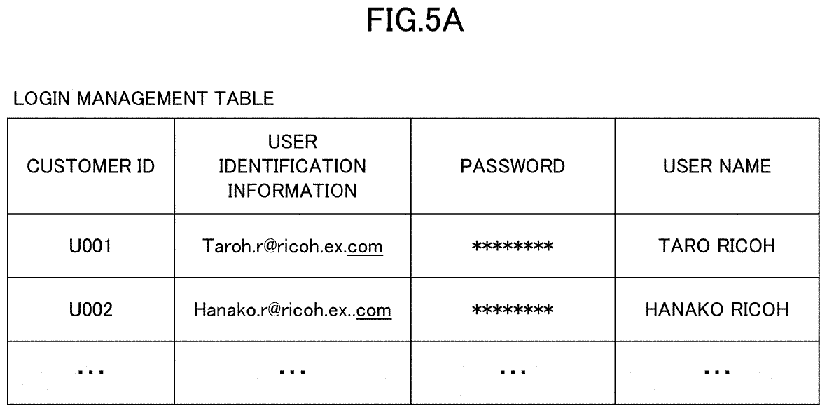

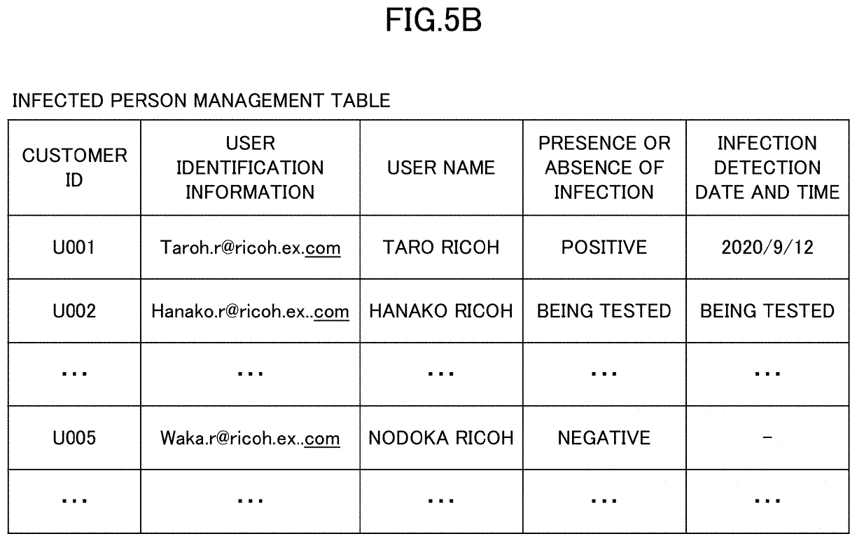

FIG. 5A is a conceptual diagram illustrating an example of a login management table according to the first embodiment. In the storage unit 2000, a login management database (DB) 2001 configured with the login management table illustrated in FIG. 5A is constructed. In this login management table, user identification information, a password, and a user name are stored and managed in association with each other for each customer ID. Among these, the customer ID is identification information associated with the customer of the company, or the like.

As user identification information, an e-mail address or the like of each user managed by a customer ID, is stored and managed. With the user identification information, for example, when the user is successfully authenticated with respect to the resource management apparatus 2, the input user account is considered to be correct by the resource management apparatus 2. This user identification information is the same as the identification information of the reservation person who has reserved the resource or of the participants of various events that take place in the resource. Therefore, based on the input user identification information, the resource management apparatus 2 can identify the reservation information in which the user who has input the user identification information is the reservation person, or the reservation information in which the user who inputs the user identification information is the participant.

The login password is the identification information associated with the user identification information at the time of login when using the communication system 1. The login password is set by the user at the time of the first activation (at the time of login), or the like. A server Uniform Resource Locater (URL) is the URL assigned to each customer of the resource management apparatus 2. The server URL may be common to multiple customers.

The user name indicates the name of each user for which user identification information is provided. The user name may be expressed by any of Japanese kanji characters, Japanese hiragana characters, Japanese katakana characters, Roman letters, numerals, or a combination thereof. According to the present embodiment, the login management table (the login management DB 2001) includes a function or means for functioning as an example of the login management means.

(Infected person management table)

FIG. 5B is a conceptual diagram illustrating an example of an infected person management table according to the first embodiment. In the storage unit 2000, an infected person management DB 2002 configured by an infection control table as illustrated in FIG. 5B is constructed. In this infected person management table, user identification information, the user name, the presence or absence of infection, and the date and time of detection of infection are stored and managed in association with each other for each customer ID. In the present embodiment, the infected person management table (the infected person management DB 2002) includes a function or means for functioning as an example of infected person management means.

Among these, the presence or absence of infection is managed as infection presence/absence information to manage whether infection or positivity has been detected, for example, with respect to Class 1 to Class 5 infectious diseases classified by the Ministry of Health, Labour and Welfare, infectious diseases such as the pandemic influenza, specified infectious diseases, new infectious diseases, or the like. For example, items such as “positive”, “being test”, “negative”, or the like are included.

The date and time of the detection of infection is managed as the date and time of the detection of infection or positivity with respect to the specific infectious disease described above, as infection detection date and time information.

### (Resource State Management Table)

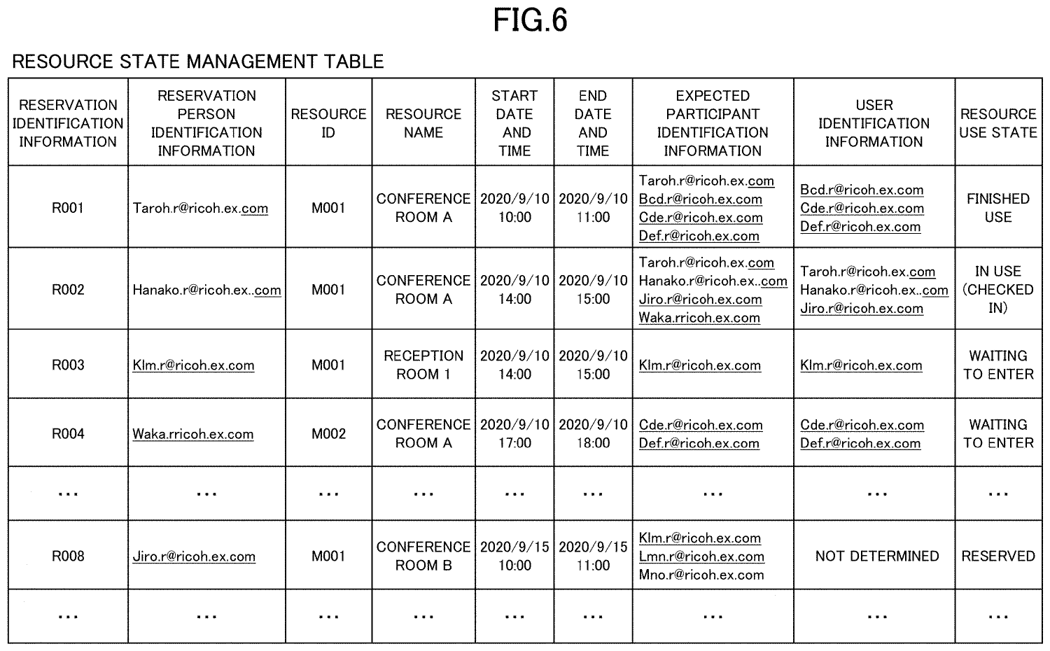

FIG. 6 is a conceptual diagram illustrating an example of a resource state information table according to the first embodiment. A resource state management DB 2003 configured with a resource state management table illustrated in FIG. 6 is constructed in the storage unit 2000. In the resource state management table, reservation person identification information, a resource ID, a resource name, a start date and time, an end date and time, expected participant identification information, user identification information, and a resource use state are stored and managed in association with each other for each piece of reservation identification information for identifying the reservation person.

Among these, the reservation identification information is identification information for identifying one record of the reservation information. Hereinafter, the same applies to identification information other than the reservation identification information. The reservation identification information is information for checking into a resource, such as a conference room, where a predetermined event takes place. The reservation identification information may be characters, a multi-digit number, or a combination thereof, that is uniquely applied. Checking in means starting to use a resource. The reservation identification information is also referred to as a reservation ID (identification). An ID is a name, a code, a character string, a numeric value, or a combination of one or more of these items, used to uniquely distinguish a particular object from multiple objects.

The reservation person identification information is provided, for example, by the account (e.g., e-mail address) of the reservation person who has reserved each resource in the floor space of the communication system 1, and Taro.r@ricoh.ex.com or the like is stored and managed.

The resource ID is identification information for identifying each resource (such as a conference room) in the in-house network. The resource name is the name of the resource associated with the resource ID. For example, a name such as

“Conference room A”, “Conference room B”, “Reception Room 1”, or the like is assigned.

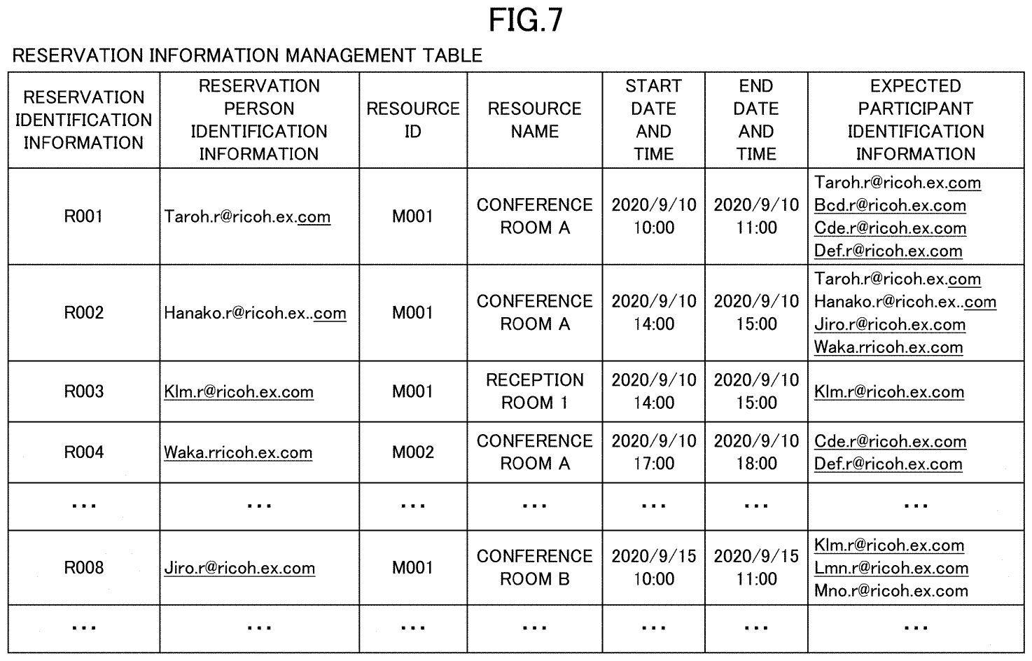

The start date and time is the date and time at which a predetermined event (a conference, business entertainment, a lecture, an exhibition, or the like) to be held at the reserved resources is to be started. The end date and time is the date and time at which a predetermined event that is to be held at the reserved resource is to be ended. The start date and time and the end date and time are updated and managed as the start date and time and the end date and time when the resource was actually used. That is, the start date and time and the end date and time managed by the reservation information management table (a reservation information management DB 3001, see FIG. 7), which will be described later, are updated according to a check in process, a check out process, or the like of the user using the reserved resource.

The expected participant identification information is the identification information of an expected participant who is scheduled to participate in a conference, and for example, the expected participant identification information is represented by an e-mail address of each expected participant.

The user identification information is the identification information of a user who participated in an event held in a predetermined resource. Among the items of the expected participant identification information, the e-mail address or the like of the user who actually participated in the event held in the predetermined resource as described above, is stored and managed as the user identification information. The reservation person identification information, the expected participant identification information, and the user identification information illustrated in FIG. 6 are all identification information issued by the reservation management apparatus 3.

The resource use state indicates the current state of the target resource. If the resource is a space such as a conference room, for example, the resource use state includes “finished use”, “in use (checked in)”, “waiting to enter”, “reserved”, or the like. Note that the resource usage state may be associated with the resource reservation status. For example, information relating to the reservation of each resource, such as statuses including reported, usage validated, waiting to check in, checked in, checked out, canceled, or the like may be registered. These usage states may be changed in association with the relationship between the current time and the start date and time or the end date and time, as well as a predetermined operation by the user (a check in process, a check out process, or the like).

In the present embodiment, the resource state management table (the resource state management DB 2003, see FIG. 6) includes a function or means for functioning as an example of resource state management means.

### <<Each Functional Configuration of Resource Management Apparatus>>

Next, each functional configuration of the resource management apparatus 2 will be described in detail. The transmitting/receiving unit 21 of the resource management apparatus 2 illustrated in FIG. 4 is mainly implemented by the processing by the CPU 201 with respect to the external device connection I/F 207 and the network I/F 209 illustrated in FIG. 2, and transmits and receives various kinds of data or information with another device or terminal (for example, the reservation management apparatus 3, the communication terminal 4, and the resource terminal 5) via the communication network 100. In the present embodiment, the transmitting/receiving unit 21 includes a function or means for functioning as an example of transmitting means or receiving means.

The acquiring unit 22 is mainly implemented by the processing by the CPU 201 illustrated in FIG. 2, and acquires the authentication information (the user identification information and the password, the administrator ID and the password) transmitted from the communication terminal 4, and registers the acquired authentication information in the login management table configuring the login management DB 2001. The acquiring unit 22 acquires reservation information relating to a predetermined resource from the reservation management apparatus 3 and registers the reservation information in the resource state management table configuring the resource state management DB 2003. In the present embodiment, the acquiring unit 22 includes a function or means for functioning as an example of acquiring means.

The clock unit 23 is mainly implemented by the processing by the CPU 201 illustrated in FIG. 2, and manages the time information in the resource management apparatus 2.

The searching/determining unit 25 is mainly implemented by the processing by the CPU 201 illustrated in FIG. 2, and searches for user identification information managed by the resource state management table (the resource state management DB 2003, see FIG. 6) and performs various determinations in the resource management apparatus 2. In the present embodiment, the searching/determining unit 25 includes a function or means for functioning as an example of searching means.

The authenticating unit 26 is mainly implemented by the processing by the CPU 501 illustrated in FIG. 2, and performs an authentication process with respect to a user based on the user identification information and the login password transmitted from the communication terminal 4.

The registering unit 28 registers the user identification information and the resource use state information representing the use state of the resource in the resource state management table (the resource state management DB 2003, see FIG. 6), which will be described later, based on the user identification information of the user who is actually using (or actually used) the conference room A, received by the transmitting/receiving unit 21. In the present embodiment, the registering unit 28 includes a function or means for functioning as an example of registering means.

The storage/reading processing unit 29 is mainly implemented by the processing by the CPU 201 illustrated in FIG. 2, and stores various kinds of data (or information) in the storage unit 2000 or reads various kinds of data (or information) from the storage unit 2000. <Functional Configuration of Reservation Management Apparatus>

As illustrated in FIG. 4, the reservation management apparatus 3 includes a transmitting/receiving unit 31, a determining unit 35, an authenticating unit 36, a reservation information managing unit 37, and a storage/reading processing unit 39. Each of these units is a function or means that is implemented by one of the elements illustrated in FIG. 2 being operated according to instructions from the CPU 301 according to a program for the reservation management apparatus 3 loaded in the RAM 303 from at least one of the ROM 302 or the HD 304.

The reservation management apparatus 3 includes a storage unit 3000 that is constructed by the ROM 302 or the HD 304 illustrated in FIG. 2. (Reservation information management table)

FIG. 7 is a conceptual diagram illustrating an example of a reservation information management table according to the first embodiment. The reservation information management DB 3001 configured by a reservation information management table illustrated in FIG. 7 is constructed in the storage unit 3000. In this reservation information management table, for each reservation identification information, reservation person identification information, a resource ID, a resource name, a start date and time, an end date and time, and expected participant identification information are stored and managed in association with each other. The above-described items are the same as the items described in the resource state management table illustrated in FIG. 6, and thus the descriptions thereof will be omitted. In the present embodiment, the reservation information management table (the reservation information management DB 3001) includes a function or means for functioning as an example of reservation information management means. However, the start date and time and the end date and time may be managed as the scheduled start date and time and the scheduled end date and time, respectively, and may be managed separately from the start date and time and the end date and time when actually used. Further, as described in the above-described resource state management table (the resource state management DB 2003, see FIG. 6), the reservation information management table may be updated in association with predetermined processing by the user.

### <<Each Functional Configuration of Reservation Management Apparatus>>

Next, each functional configuration of the reservation management apparatus 3 will be described in detail. The transmitting/receiving unit 31 of the reservation management apparatus 3 illustrated in FIG. 4 is mainly implemented by processing of the CPU 301 with respect to the external device connection I/F 307 and the network I/F 309 illustrated in FIG. 2, and transmits and receives various kinds of data (or information) with the resource management apparatus 2 via the communication network 110 constructed outside the firewall 105. The transmitting/receiving unit 31 receives the login authentication request and the setting of the reservation when using the free address of the resource terminal 5.

The determining unit 35 is mainly implemented by the processing by the CPU 301 illustrated in FIG. 3, and performs various determinations in the reservation management apparatus 3.

The authenticating unit 36 is mainly implemented by the processing by the CPU 301 illustrated in FIG. 3, and authenticates the user based on the authentication information (for example, the user identification information and the password) stored in the storage unit 3000. The authentication may be performed with an integrated circuit (IC) card or biometric authentication information.

The reservation information managing unit 37 is mainly implemented by the processing by the CPU 301 illustrated in FIG. 3, and manages the reservation information registered by the user. The reservation information managing unit 37 reads various kinds of reservation information from the reservation information management table (the reservation information management DB 3001) via the storage/reading processing unit 39 which will be described later and transmits the read information to the resource management apparatus 2 via the transmitting/receiving unit 31.

The storage/reading processing unit 39 is mainly implemented by the processing by the CPU 301 illustrated in FIG. 3, and stores various kinds of data (or information) in the storage unit 3000 or reads various kinds of data (or information) from the storage unit 3000.

### <Functional Configuration of Communication Terminal>

As illustrated in FIG. 4, the communication terminal 4 includes a transmitting/receiving unit 41, an accepting unit 42, a clock unit 43, a display control unit 44, a capturing/acquiring unit 46, an application activating unit 47, a registering unit 48, and a storage/reading processing unit 49. Each of these functional units is a function or means implemented by one of the elements illustrated in FIG. 3 being operated by instructions from the CPU 401 according to a program for the communication terminal 4 loaded from at least one of the ROM 402 or the EEPROM 404 to the RAM 403. The communication terminal 4 includes a storage unit 4000 that is constructed by the ROM 402 or the EEPROM 404 illustrated in FIG. 3.

### <<Each Functional Configuration of Communication Terminal>>

Next, each functional configuration of the communication terminal 4 will be described in detail. The transmitting/receiving unit 41 of the communication terminal 4 illustrated in FIG. 4 is mainly implemented by the processing by the CPU 401 with respect to the long range communication circuit 412 and the external device connection I/F 419 illustrated in FIG. 3, and transmits and receives various kinds of data (or information) with the resource terminal 5 via the communication network 100 constructed inside the firewall 105. The transmitting/receiving unit 41 transmits and receives various kinds of data (or information) with the resource management apparatus 2 via the communication network 110 constructed outside the firewall 105. In the present embodiment, the transmitting/receiving unit 41 includes a function or means for functioning as an example of transmitting means and receiving means.

The accepting unit 42 is mainly implemented by the processing by the CPU 401 to the touch panel 421 illustrated in FIG. 3, and accepts the user input in the authentication processing at the time of starting to use the resource or the like, the check in processing by the user who uses the resource, or the like. In the present embodiment, the accepting unit 42 includes a function or means for functioning as an example of the reception means.

The clock unit 43 is mainly implemented by the processing by the CPU 401 illustrated in FIG. 3, and manages the time information in the communication terminal 4.

The display control unit 44 is mainly implemented by the processing by the CPU 401 with respect to the display 418 illustrated in FIG. 3, and displays the user authentication screen, the QR code capture screen, the check in list display screen, the infected person (positive person) registration screen, or the like on the display 418. In the present embodiment, the display control unit 44 includes a function or means for functioning as an example of display control means.

The capturing/acquiring unit 46 is mainly implemented by the processing by the CPU 401 with respect to the CMOS sensor 413 and the imaging element I/F 414 illustrated in FIG. 3. The capturing/acquiring unit 46 captures a subject (a one-dimensional code such as a bar code or a two-dimensional code such as a QR code) displayed on the display 518 of the resource terminal 5 and acquires predetermined code information embedded in the code. In the present embodiment, the capturing/acquiring unit 46 includes a function or means for functioning as an example of the capturing/acquiring means.

The application activating unit 47 is mainly implemented by the processing by the CPU 401 illustrated in FIG. 3, and activates a user authentication application installed in the storage unit 4000 of the communication terminal 4 to perform user authentication and code capturing when using a predetermined resource, an infected person registration application to register an infected person when infected by a particular infection, or the like.

The registering unit 48 is mainly implemented by the processing by the CPU 401 illustrated in FIG. 3, and acquires code information from a subject (a one-dimensional code such as a bar code or a two-dimensional code such as a QR code) captured by the capturing/acquiring unit 46, and registers the acquired information. Further, the registering unit 48 registers, in the storage means, user identification information, a password, or the like as authentication information transmitted by the resource management apparatus.

The storage/reading processing unit 49 is mainly implemented by the processing by the CPU 401 illustrated in FIG. 3, and stores various kinds of data (or information) in the storage unit 4000 or reads various kinds of data (or information) from the storage unit 4000.

### <Functional Configuration of Resource Terminal>

As illustrated in FIG. 4, the resource terminal 5 includes a transmitting/receiving unit 51, an accepting unit 52, a generating unit 53, a display control unit 54, a terminal authenticating unit 56, and a storage/reading processing unit 59. Each of these units is a function or means implemented by one of the elements illustrated in FIG. 3 being operated by instructions from the CPU 501 according to a program for the resource terminal 5 loaded in the RAM 503 from at least one of the ROM 502 or the EEPROM 504.

The resource terminal 5 includes a storage unit 8000 that is constructed by the ROM 502 or the EEPROM 504 illustrated in FIG. 3.

### <<Each Functional Configuration of Resource Terminal>>

Next, each functional configuration of the resource terminal 5 will be described in detail. The transmitting/receiving unit 51 of the resource terminal 5 illustrated in FIG. 4 is mainly implemented by the processing by the CPU 501 with respect to the long range communication circuit 512 and the external device connection I/F 519 illustrated in FIG. 3, and transmits and receives various kinds of data (or information) with the reservation management apparatus 3 and the resource management apparatus 2, via the communication network 100.

The accepting unit 52 is implemented mainly by the processing by the CPU 501 and the processing by the touch panel 521 illustrated in FIG. 3, and accepts various selections or inputs from a user. In addition to the touch panel 521, an operation button or the like may be used as another input means.

The generating unit 53 is mainly implemented by the processing of the CPU 501 and the display 518 illustrated in FIG. 3, and generates a code including the reservation identification information transmitted by the resource management apparatus 2. The code generated at this time is, for example, a one-dimensional code or a two-dimensional code, and specifically, various codes such as bar codes, QR codes or the like are generated. Further, the two-dimensional code may be a color code. The data represented by these codes is data encrypted by public key encryption or private key encryption. The above-described method for generating various codes is publicly known, and, therefore, detailed descriptions thereof will be omitted here.

The display control unit 54 is mainly implemented by the processing by the CPU 501 and the display 518 illustrated in FIG. 3, and performs control for displaying various images, characters, predetermined codes (QR codes), or the like on the display 518. In the present embodiment, an image such as an entry screen, a QR code, or the like is displayed on the display 518 at the time of check in at a predetermined resource or the like.

The terminal authenticating unit 56 is mainly implemented by the processing by the CPU 501 illustrated in FIG. 3. For example, the terminal authenticating unit 56 authenticates the login to the terminal application based on whether the input password is correct.

The storage/reading processing unit 59 is mainly implemented by the processing by the CPU 501 illustrated in FIG. 3, and stores various kinds of data (or information) in a storage unit 5000 or reads various kinds of data (or information) from the storage unit 5000.

### [Processing or Operation of Embodiment]

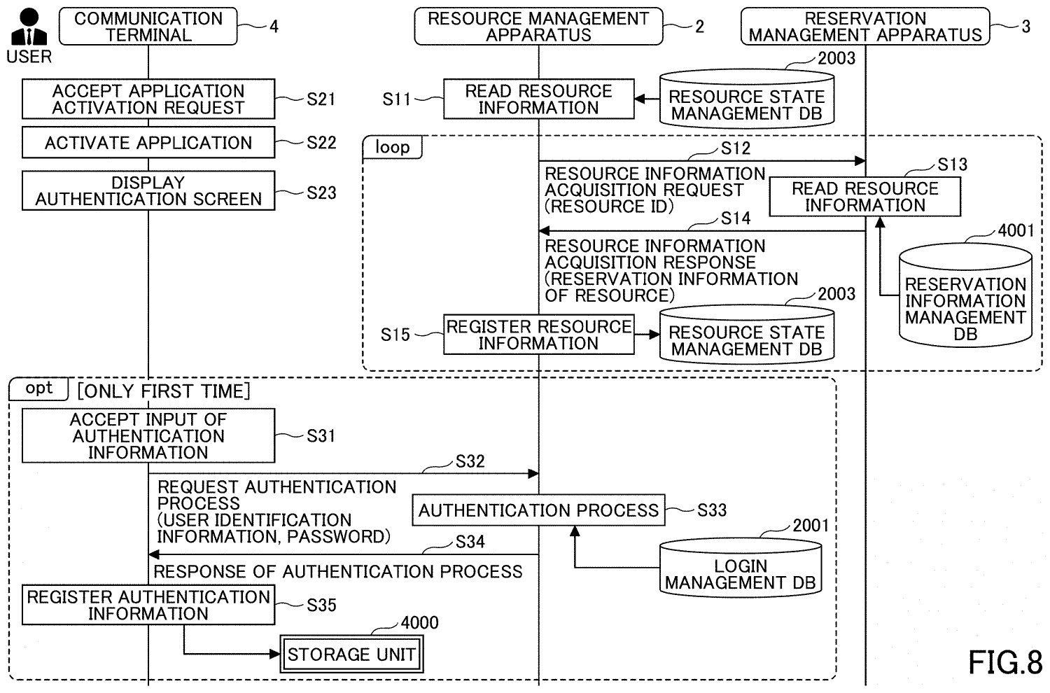

Next, the processing or operation in the communication system 1 according to the first embodiment will be described with reference to FIGS. 8 to 19.

### <Authentication Process>

First, the authentication process in the communication terminal 4 will be described with reference to FIG. 8. FIG. 8 is a sequence diagram illustrating an example of an authentication process according to the first embodiment. First, in step S11, the storage/reading processing unit 29 of the resource management apparatus 2 reads the reservation identification information (R001, R002, from the resource state management table (the resource state management DB 2003, see FIG. 6).

Next, in step S12, the transmitting/receiving unit 21 of the resource management apparatus 2 transmits a resource information acquisition request to the reservation management apparatus 3. Therefore, the transmitting/receiving unit 31 of the reservation management apparatus 3 receives the resource information acquisition request transmitted by the resource management apparatus 2. At this time, the resource information acquisition request includes the resource ID corresponding to the reservation identification information. In the present embodiment, the transmitting/receiving unit 21 includes a function or means for functioning as an example of the transmitting means.

Subsequently, in step S13, the storage/reading processing unit 39 of the reservation management apparatus 3 reads the reservation information of the corresponding resource by searching the reservation information management table (the reservation information management DB 3001, see FIG. 7) using the resource ID received by the transmitting/receiving unit 31 as the search key.

Subsequently, in step S14, the transmitting/receiving unit 31 transmits a resource information acquisition response to the resource management apparatus 2 as a response to the resource information acquisition request. Therefore, the transmitting/receiving unit 21 of the resource management apparatus 2 receives the resource information acquisition response transmitted by the transmitting/receiving unit 31 of the reservation management apparatus 3. The resource information acquisition response includes, for example, reservation identification information, reservation person identification information, a resource ID, a resource name, the start date and time, the end date and time, and expected participant identification information, which are treated as reservation information. In the present embodiment, the transmitting/receiving unit 31 includes a function or means for functioning as an example of the receiving means.

In step S15, the acquiring unit 22 of the resource management apparatus 2 registers the reservation information received by the transmitting/receiving unit 21 in the resource state management table (the resource state management DB 2003, see FIG. 6). Accordingly, the acquired reservation information relating to each resource is registered and managed in the resource state management table (the resource state management DB 2003, see FIG. 6). The resource information does not change frequently, and, therefore, the resource management apparatus 2 and the reservation management apparatus 3 may perform the above-described processes from step S12 to step S15 as a loop process. Specifically, the acquiring unit 22 may acquire the reservation information at a low frequency, such as once a day. The acquiring unit 22 may acquire the reservation information, from the reservation management apparatus 3, periodically or when the reservation information is updated.

In the communication system 1 according to the present embodiment, when the above-described processes of step S12 and step S14 are performed, another device or the like may exist between the resource management apparatus 2 (resource management apparatus) and the reservation management apparatus 3 (reservation management apparatus). That is, each piece of information transmitted and received between the resource management apparatus 2 and the reservation management apparatus 3 may be transmitted and received once via another device. The above-described configurations and processing methods can be applied to other processing steps below.

### <<Application Activation Process>>

Subsequently, in step S21, the accepting unit 42 of the communication terminal 4 accepts a request to activate an application operated by a reservation person who has reserved the resource or a user who uses the resource.

Accordingly, in step S22, the application activating unit 47 of the communication terminal 4 activates, for example, a browser (exclusive-use application).

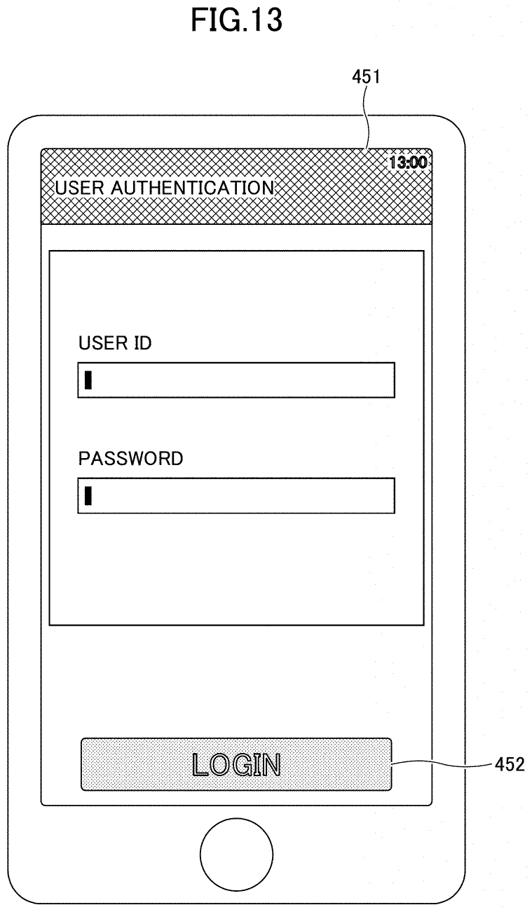

When the exclusive-use application is activated, the display control unit 44 displays the authentication screen on the display 418 (step S23). FIG. 13 is a diagram illustrating an example of the authentication screen according to the first embodiment. In FIG. 13, the display control unit 44 displays an example of a user authentication screen 451 (for example, a “user ID” input portion, a “password” input portion, and a “login” button) on the display 418 of the communication terminal 4.

In response to the process in step S23, the reservation person or the user (hereinafter, referred to as a “user A”) performs an operation to log in to the resource management apparatus 2. Specifically, the user A inputs the user ID and password and operates (presses or taps) a login button 452 to perform the login process. Accordingly, in step S31, the accepting unit 42 accepts the input of the authentication information. For example, the user identification information and password are used as the authentication information. Here, it is assumed that the authentication is successful.

After accepting the input of the authentication information in step S31, in step S32, the transmitting/receiving unit 41 transmits an authentication process request to the resource management apparatus 2. Accordingly, the transmitting/receiving unit 21 of the resource management apparatus 2 receives the authentication process request transmitted from the communication terminal 4. The authentication process request includes the user identification information and the password.

Subsequently, in step S33, the storage/reading processing unit 29 of the resource management apparatus 2 reads the corresponding user identification information, the login password, and the user name by searching the login management table (the login management DB 2001, see FIG. 5A) using the received user identification information or login password as the search key, as the reading process of the login information. Accordingly, the authenticating unit 26 performs the process of authenticating the user. In the present embodiment, the storage/reading processing unit 29 includes a function or means for functioning as an example of the storage/reading means.

When user authentication is successful, in step S34, the transmitting/receiving unit 21 transmits an authentication process response including the user identification information of the user, to the communication terminal 4. Accordingly, the transmitting/receiving unit 41 of the communication terminal 4 receives the authentication process response including the user identification information of the user transmitted by the resource management apparatus 2.

In step S35, the communication terminal 4 that has received the authentication process response including the user identification information of the user, stores the user identification information and the password included in the received authentication process response, in the storage unit 4000.

In the communication system 1 according to the present embodiment, when the above-described processes of step S24 and step S26 are performed, another device or the like may exist between the resource management apparatus 2 (resource management apparatus) and the communication terminal 4 (communication terminal). That is, each piece of information transmitted and received between the resource management apparatus 2 and the communication terminal 4 may be transmitted and received once via another device. The above-described configurations and processing methods can be applied to other processing steps below.

### <<Check in Process>>

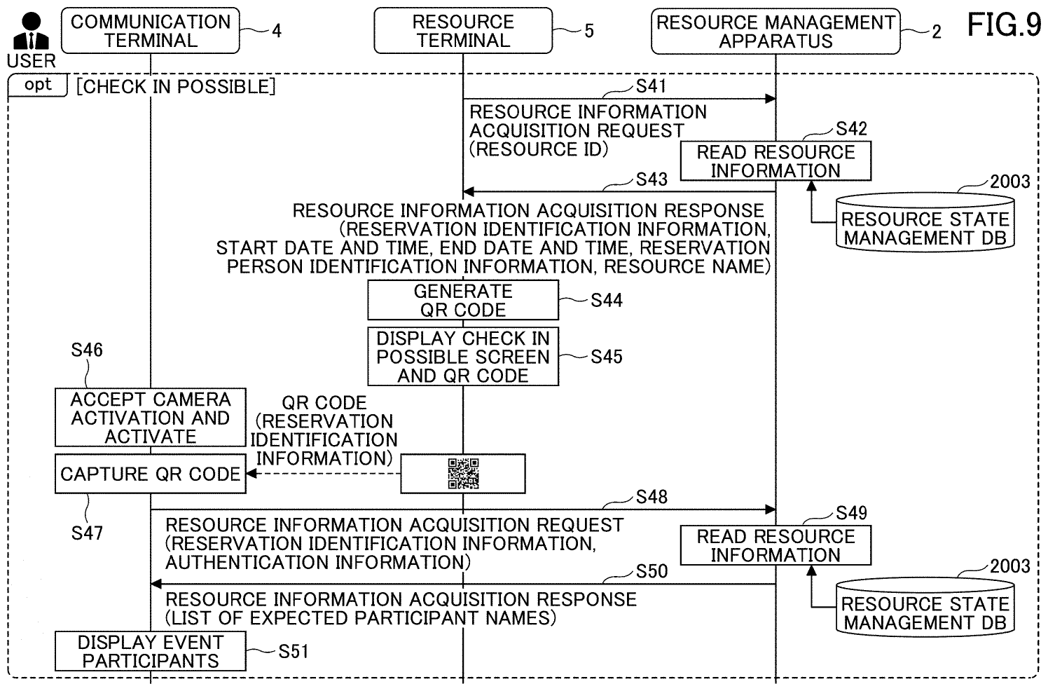

Next, the check in process of checking into the resource by the user A will be described. FIG. 9 is a sequence diagram illustrating an example of a check in process performed with a QR code according to the first embodiment. In step S41, the transmitting/receiving unit 51 of the resource (for example, the conference room) to be used by the user A transmits a resource information acquisition request to the resource management apparatus 2. Accordingly, the transmitting/receiving unit 21 of the resource management apparatus 2 receives the resource information acquisition request transmitted by the resource terminal 5. At this time, the resource information acquisition request includes the resource ID of the resource that is scheduled to be used. Here, with regard to the timing when step S41 is executed, the resource management apparatus 2 may send a push report to the resource terminal 5 upon being triggered by the response of the authentication process in step S34 described above. Further, the resource terminal 5 may periodically transmit the resource information acquisition request to the resource management apparatus 2 executed in step S41.

In step S42, the storage/reading processing unit 29 of the resource management apparatus that has received the resource information acquisition request, reads the corresponding reservation person identification information, resource name, start date and time, end date and time, and expected participant identification information by searching the resource state management table (the resource state management DB 2003, see FIG. 6) using the received resource ID as the search key.

Subsequently, in step S43, the transmitting/receiving unit 21 transmits, to the resource terminal 5, the reservation person identification information, the resource name, the start date and time, the end date and time, and the expected participant identification information read in step S42. Accordingly, the transmitting/receiving unit 51 of the resource terminal 5 receives the reservation person identification information, the resource name, the start date and time, the end date and time, and the expected participant identification information transmitted by the resource management apparatus 2.

In the communication system 1 according to the present embodiment, when the above-described processes of step S42 and step S43 are performed, another device or the like may exist between the resource management apparatus 2 (resource management apparatus) and the resource terminal 5 (resource terminal). That is, each piece of information transmitted and received between the resource management apparatus 2 and the resource terminal 5 may be transmitted and received once via another device. The above-described configuration and processing methods can be applied to other processing steps below.

Next, in step S44, the generating unit 53 of the resource terminal 5 generates a predetermined code (for example, a QR code) relating to the resource terminal 5 in which the received reservation person identification information is embedded. As described above, the method for generating various codes such as bar codes and QR codes is publicly known, and therefore, the detailed description thereof will be omitted.

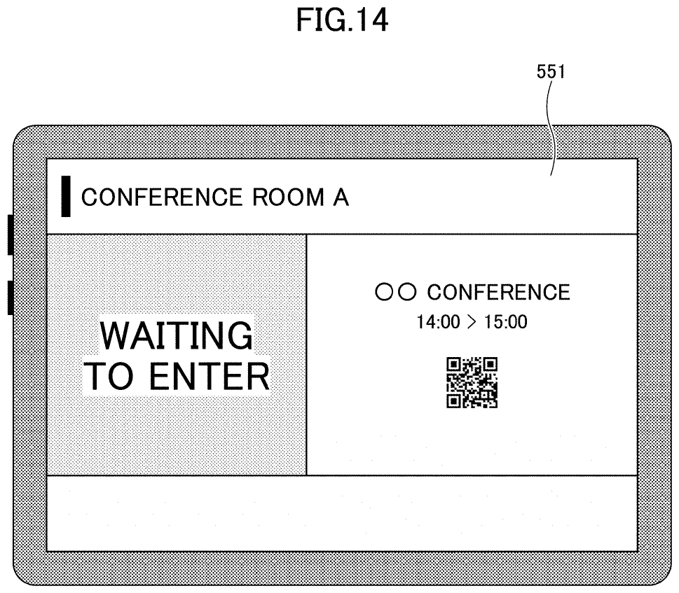

Subsequently, in step S45, the display control unit 54 displays a check in possible screen 551 as illustrated in FIG. 14. FIG. 14 is a diagram illustrating an example of a check in possible screen on the resource terminal according to the first embodiment. In FIG. 14, the display control unit 44 displays an example of the check in possible screen 551 (for example, “conference room A”, “waiting to enter”, “XX conference”, “14:00>15:00”, and “QR code”) on the display 518 of the resource terminal 5.

When the user confirms that the check in possible screen 551 is displayed on the display 518 of the resource terminal 5, the user performs an operation of activating the camera of the communication terminal 4 possessed by the user. Accordingly, in step S46, the CPU 401, which is the subject of processes of the capturing/acquiring unit 46 of the communication terminal 4, activates the camera by controlling the CMOS sensor 413 and the imaging element I/F 414.

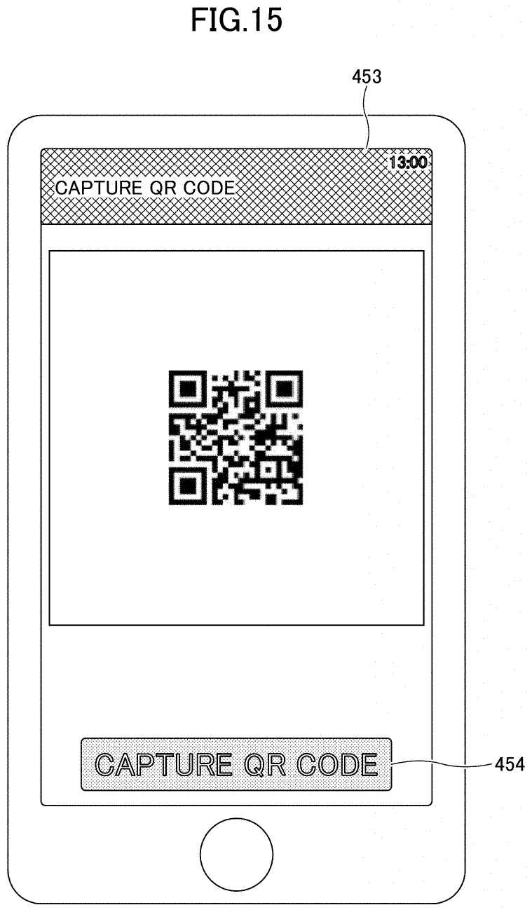

The user A who has performed the operation of activating the camera, approaches the check in possible screen 551 and operates a QR code capturing button 454 (see FIG. 15) to capture the QR code displayed on the screen. Accordingly, in step S47, the capturing/acquiring unit 46 captures the QR code according to the operation of the user A and acquires predetermined code information (for example, reservation identification information) embedded in the QR code. That is, the capturing/acquiring unit 46 acquires code information embedded in a predetermined code by capturing a predetermined code (QR code) relating to predetermined reservation information displayed by the resource terminal 5 capable of communicating with the resource management apparatus 2. At this time, the display control unit 44 causes the display 418 of the communication terminal 4 to display a QR code capturing screen 453 as illustrated in FIG. 15. FIG. 15 is a diagram illustrating an example of a QR code capturing screen according to the first embodiment.

After capturing the QR code, in step S48, the transmitting/receiving unit 41 transmits the resource information acquisition request to the resource management apparatus 2. Accordingly, the transmitting/receiving unit 21 of the resource management apparatus 2 receives the resource information acquisition request transmitted by the communication terminal 4. At this time, the resource information acquisition request includes the reservation identification information acquired by the capturing/acquiring unit 46 and the authentication information received in step S24.

After receiving the resource information acquisition request, in step S49, the storage/reading processing unit 29 of the resource management apparatus 2 reads the corresponding expected participant identification information by referring to the resource state management table (the resource state management DB 2003, see FIG. 6) using the reservation identification information as the search key.

Subsequently, in step S50, the transmitting/receiving unit 21 transmits the expected participant identification information that has been read, as a resource information acquisition response, to the communication terminal 4. Accordingly, the transmitting/receiving unit 41 of the communication terminal 4 receives the expected participant identification information as the resource information acquisition response transmitted from the resource management apparatus 2.

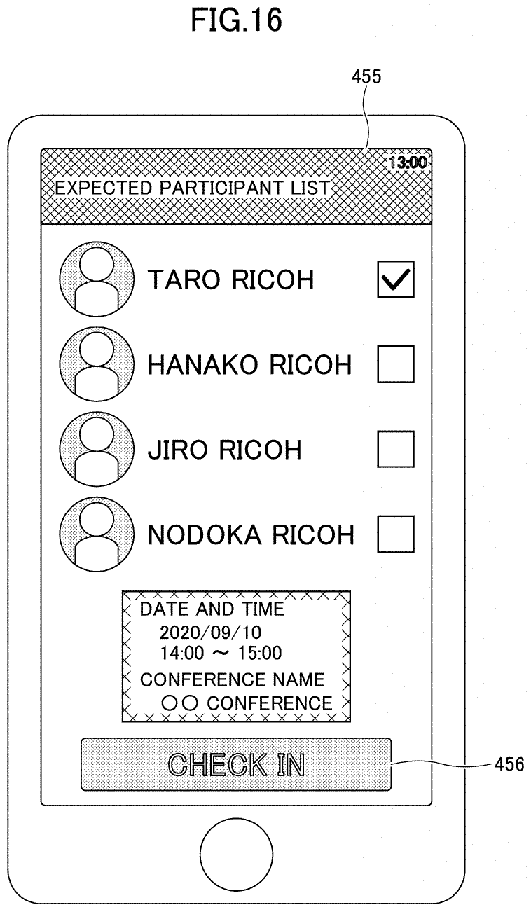

Thereafter, in step S51, the display control unit 44 of the communication terminal 4 displays an expected participant list screen 455 as illustrated in FIG. 16 on the display 418 of the communication terminal 4. FIG. 16 is a diagram illustrating an example of the expected participant list display screen according to the first embodiment. That is, the display control unit 44 displays, on the display 418, a list of expected participant names corresponding to the expected participant identification information received by the transmitting/receiving unit 21.

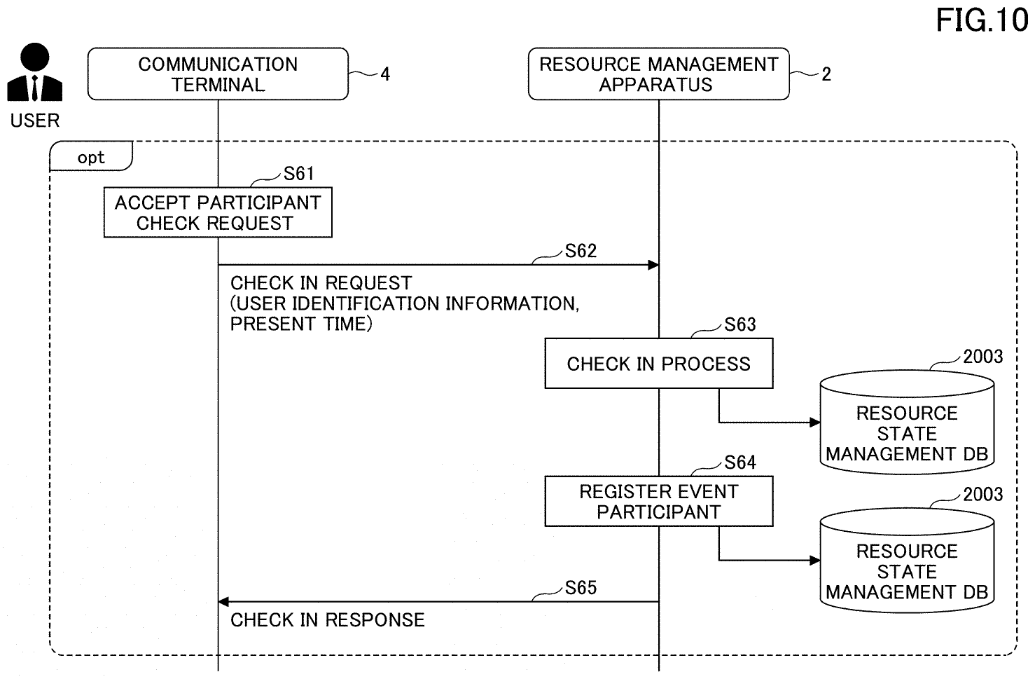

FIG. 10 is a sequence diagram illustrating an example of a check in process performed with the QR code according to the first embodiment. The user A inputs a check mark (tick mark) with respect to the participant who will actually use the reserved resource, in the expected participant list screen 455 illustrated in FIG. 16. Accordingly, in step S61, the accepting unit 42 accepts the participant check request according to the operation of the user A. Specifically, if the expected participant displayed on the expected participant list screen 455 will actually participate in the use of the resource, the user A inputs a check mark (tick mark) in the check box next to the corresponding expected participant in the expected participant list. The user A performs a check in process by operating (pressing or tapping) a check in button 456 when the user A has completed inputting check marks for all participants. That is, the accepting unit 42 selects and accepts the users who will use the reserved resource from the list of expected participants names displayed on the display 418.

After the check in process is performed by the user A, in step S62, the transmitting/receiving unit 41 transmits the check in request as a start request when using the reserved resource, to the resource management apparatus 2. Accordingly, the transmitting/receiving unit 21 of the resource management apparatus 2 receives the check in request transmitted from the communication terminal 4. At this time, the check in request includes user identification information indicating the information of each participant for which a check mark has been input in the expected participant list illustrated in FIG. 16 and the current time information indicating the current time. That is, the transmitting/receiving unit 41 transmits the user identification information of the user A currently using the reserved target resource associated with the code information (the reservation identification information) acquired by the capturing/acquiring unit 46 and embedded in the QR code, to the resource management apparatus 2, as a start request for starting use of the resource.

In step S63, the resource management apparatus 2 that has received the check in request performs the check in process. Specifically, the storage/reading processing unit 29 writes “in use (checked in)” as the resource use state of the reserved resource managed in the resource state management table (the resource state management DB 2003, see FIG. 6).

Subsequently, in step S64, the registering unit 28 registers an event participant participating in a predetermined event (a conference or the like) that is held using the reserved resource, via the storage/reading processing unit 29. Specifically, in the example illustrated in FIGS. 6 and 16 (in the case where the conference room A is used as the resource), it is illustrated that the registering unit 28 has registered, via the storage/reading processing unit 29, the expected participants of the resource (the conference room A) reserved by the reservation identification information of R002, i.e., Taro.r@ricoh.ex.com, Hanako.r@ricoh.ex.com, Jiro.r@ricoh.ex.com, and Waka.r@ricoh.ex.com, as the user identification information of the users who have actually used the conference room A, i.e., Taro.r@ricoh.ex.com, Hanako.r@ricoh.ex.com, Jiro.r@ricoh.ex.com, Waka.r@ricoh.ex.com. That is, when the user identification information of the users who have actually used the conference room A is received by the transmitting/receiving unit 21, the registering unit 28 registers the user identification information and the resource use state information in the resource state management table (the resource state management DB 2003, see FIG. 6). Specifically, the registering unit 28 registers the user identification information and the resource use state information representing the use state of the resource in the resource state management table (the resource state management DB 2003, see FIG. 6), based on the user identification information of the users who have actually used the conference room A, received by the transmitting/receiving unit 21 via the storage/reading processing unit 29. At this time, the storage/reading processing unit 29 may change the start date and time to the date and time when the check in process was actually performed.

After executing the process in step S64, in step S65, the transmitting/receiving unit 21 transmits a check in response to the communication terminal 4. Accordingly, the transmitting/receiving unit 41 of the communication terminal 4 receives the check in response transmitted from the resource management apparatus 2.

In the communication system 1 according to the present embodiment, when the above-described processes of step S48 and step S50 and the processes of step S62 and step S65 are performed, another device or the like may exist between the resource management apparatus 2 (resource management apparatus) and the communication terminal 4 (communication terminal). That is, each piece of information transmitted and received between the resource management apparatus 2 and the communication terminal 4 may be transmitted and received once through another device. The above-described configurations and processing methods can be applied to other processing steps below.

### <<Infected Person Registration Process>>

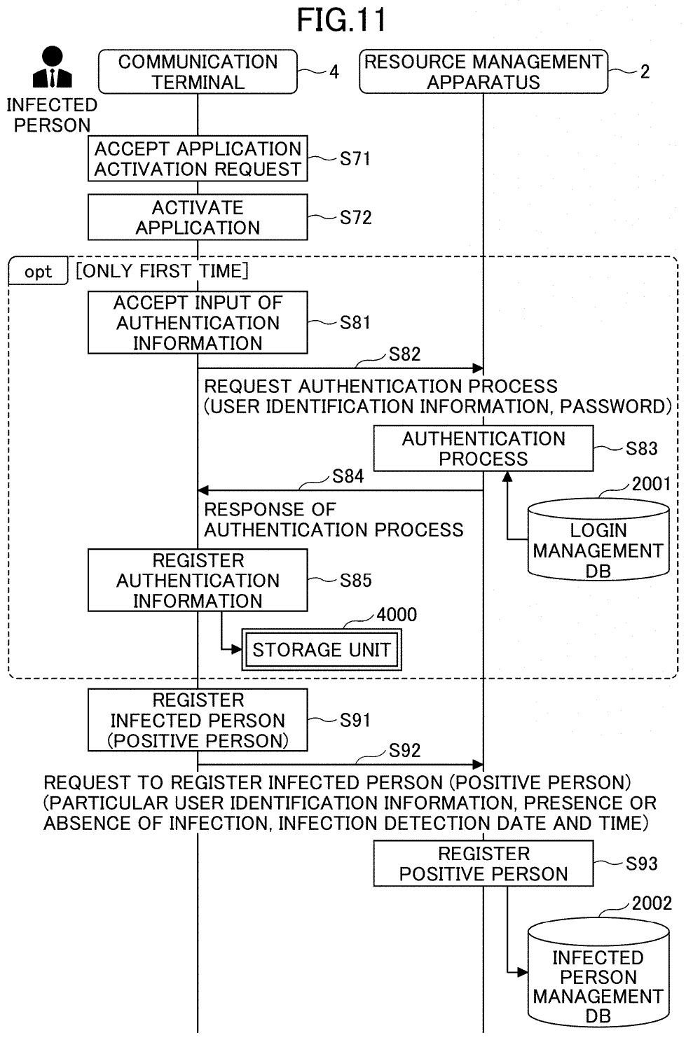

Next, the process when a particular user, among the users who have used the above-described conference room A, is infected with a particular infectious disease, will be described. FIG. 11 is a sequence diagram illustrating an example of a process of registering an infected person according to the first embodiment. In FIG. 11, a user B (for example, Taro Ricoh, user identification information: Taro.r@ricoh.ex.com) who has been infected with a particular infectious disease performs an operation of activating a particular application (infected person registration application) to register an infected person using the communication terminal 4 of his own (an infected person terminal; hereinafter referred to as an infected person terminal as a matter of convenience). Accordingly, the accepting unit 42 of the infected person terminal accepts an operation of activating a particular application for registering the infected person (step S71), and after the accepting unit 42 accepts the operation of activating the application, in step S72, the application activating unit 47 activates the infected person registration application.

Subsequently, the user B performs an authentication process from steps S81 to S85 using the infected person terminal that he/she possesses. The processes of the steps S81 to S85 are the same as those of the above-described steps S31 to S35, and thus the description thereof will be omitted.

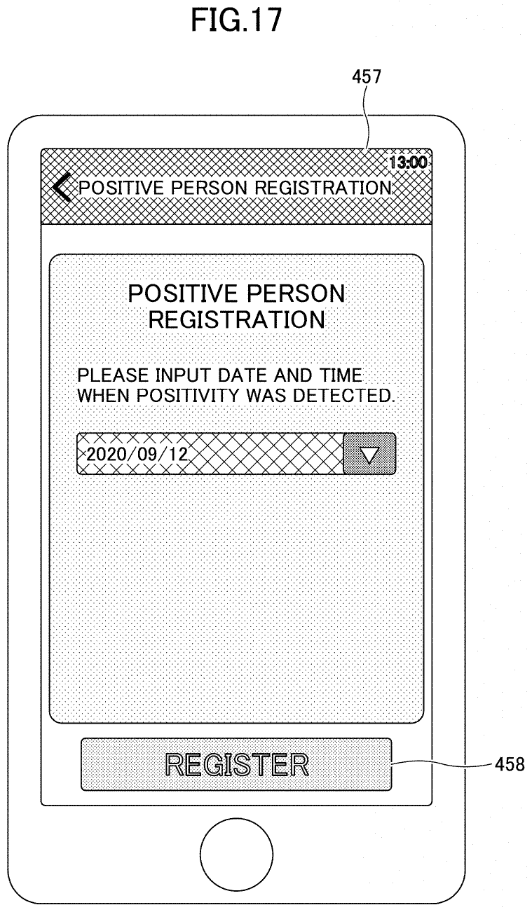

After the authentication process is performed by the user B, the registering unit 48 of the infected person terminal performs the process of registering the infected person (the positive person) (step S91). In the present embodiment, the infected person (a positive person) is referred to as an “infected person” as a matter of convenience. That is, a positive person is regarded as an example of an infected person as a matter of fact. The infected person is registered on an infected person registration screen 457 as illustrated in FIG. 17, which is activated by the application activating unit 47, by the user B inputting “the date and time when the positivity is detected” by a scrolling and selecting operation or the like and operating a registration button 458. FIG. 17 is a diagram illustrating an example of an infected person registration screen according to the first embodiment.

In step S92, after the process of registering the infected person is performed by the user B, the transmitting/receiving unit 41 transmits an infected person registration request to the resource management apparatus 2. Accordingly, the transmitting/receiving unit 21 of the resource management apparatus 2 receives the infected person registration request transmitted from the infected person terminal. At this time, the infected person registration request includes the user identification information of the user B who is the infected person and the date and time when the infection was detected (e.g., 2020/09/12). At this time, the user identification information of the user B, that is, Taro.r@ricoh.ex.com, is treated as an example of particular user identification information for identifying a particular user.

After receiving the infected person registration request, the registering unit 28 of the resource management apparatus 2 registers the user name corresponding to the user identification information, the presence or absence of infection (in this case, “positive”), and the date and time of the detection of infection (step S93).

### <<Contact Person Reporting Process>>

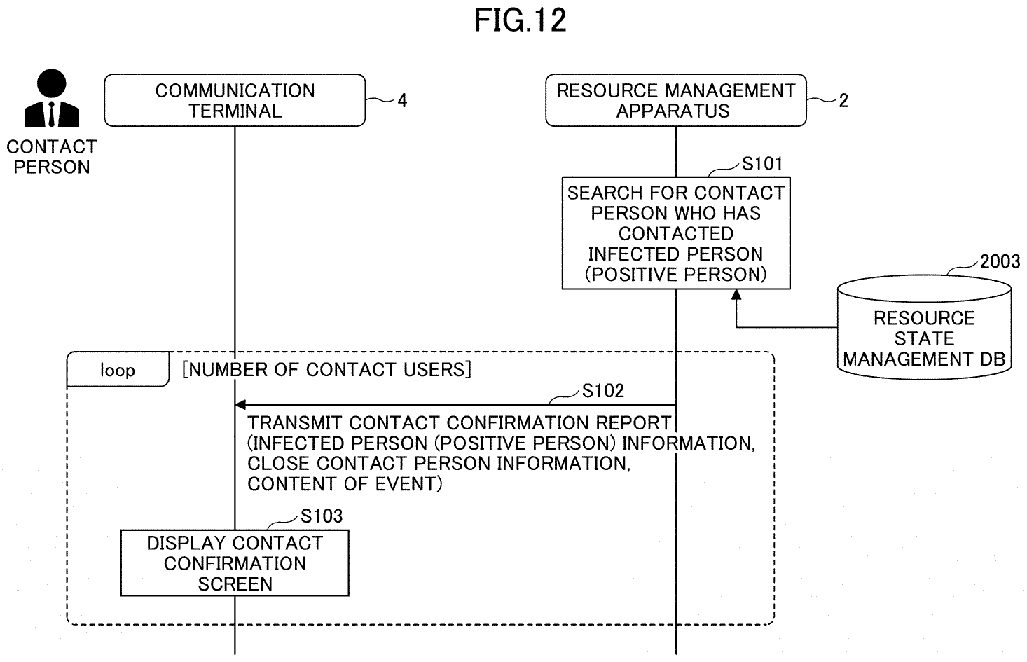

Next, a process of searching for another user who has contacted an infected person by the resource management apparatus 2 will be described. FIG. 12 is a sequence diagram illustrating an example of a process of sending a report to a contact person according to the first embodiment. As illustrated in

FIG. 12, the searching/determining unit 25 of the resource management apparatus 2 reads the user identification information of users who have used the same resource as the user B by searching the resource state management table (the resource state management DB 2003, see FIG. 6) using the user identification information: Taro.r@ricoh.ex.com of the infected user B as the search key. Subsequently, among the user identification information that has been read, the searching/determining unit 25 further searches for the reservation of resources used within a predetermined period before the date when the infection was detected (for example, 2020/09/12). In this case, the searching/determining unit 25 searches the resource state management table (the resource state management DB 2003, see FIG. 6) and determines that the users represented by the user identification information of Hanako.r@ricoh.ex.com and Jiro.r@ricoh.ex.com who have participated in the conference held in the conference room A represented by the reservation identification information R002 on 2020/09/10 14:00-15:00, are contact persons who have contacted infected person, that is, the user B (step S101).

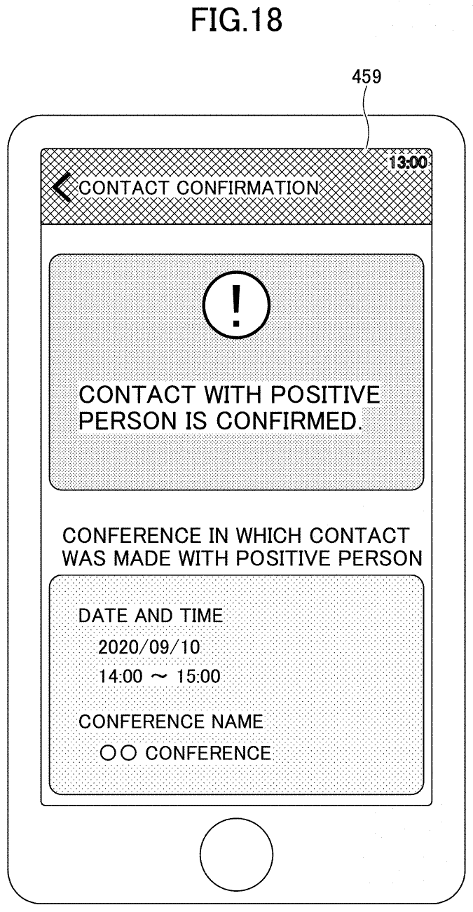

Subsequently, in step S102, the transmitting/receiving unit 21 transmits a contact confirmation e-mail as a contact confirmation report illustrated in FIG. 18, to the communication terminal (the contact person terminal) possessed by the user who is determined to be a contact person (a close contact person). Accordingly, the transmitting/receiving unit 41 of the communication terminal 4 receives the contact confirmation e-mail transmitted by the resource management apparatus 2. At this time, the transmitted contact confirmation e-mail includes information on the infected person (the positive person), information on the close contact persons, and information on the event in which the close contact is suspected. The information on the infected person (the positive person), information on the close contact persons, and information on the event include e-mail addresses and names of the infected person (the positive person) and close contact persons, as well as details of the event (e.g., date and time, conference name, or the like) held at the used resource.

Subsequently, in step S103, the display control unit 44 displays, for example, the contact confirmation screen illustrated in FIG. 18 based on the information of the infected person (the positive person) and the information on the close contact persons included in the received contact confirmation e-mail. FIG. 18 is a diagram illustrating an example of a contact confirmation screen according to the first embodiment. The transmitting/receiving unit 21 repeats the process of step S102 by the number of the contact persons. At this time, the transmitting/receiving unit 21 uses the e-mail address indicated in the user identification information of each contact person, as the destination that is the communication terminal 4 possessed by each contact person.

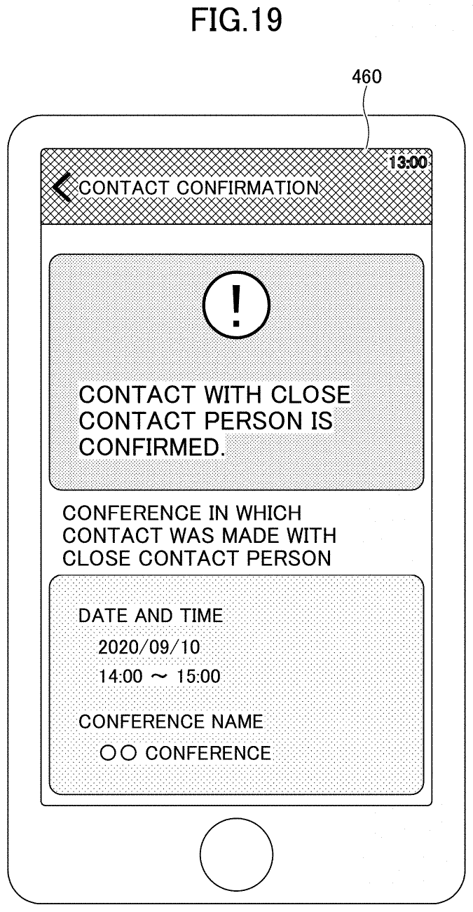

On the other hand, FIG. 19 is a diagram illustrating another example of the contact confirmation screen according to the first embodiment.

In the present embodiment, the searching/determining unit 25 may define a user who has contacted a user who has contacted the infected person, on another date and time and at another place, as a “close contact person”, and search for a corresponding user. Specifically, the searching/determining unit 25 first determines that each user represented by the user identification information of Hanako.r@ricoh.ex.com and Jiro.r@ricoh.ex.com described above, has contacted an infected person, i.e., the user B (Taro Ricoh) represented by the user identification information of Taro.r@ricoh.ex.com. Further, the searching/determining unit 25 may determine that another user who has contacted at least one of the users represented by the user identification information of Hanako.r@ricoh.ex.com and Jiro.r@ricoh.ex.com, within a predetermined period before and after (for example, about one week before and after) the date and time of detection of infection (for example, 2020/09/12), is a close contact person. As described above, when the searching/determining unit 25 searches the resource state management table (the resource state management DB 2003, see FIG. 6), and a user determined to be a close contact person is found, the transmitting/receiving unit 21 transmits the contact confirmation report illustrated in FIG. 19 to the communication terminal 4 possessed by each user determined to be a close contact person. The above-described predetermined period may be appropriately modified according to the characteristics of the infective disease (e.g., the survival period of the virus, or the like).

However, the resource management apparatus 2 does not apply the present embodiment to a remote participant who has participated remotely in an event held at the same resource at the same date and time. This is because remote participants do not use the same resource at the same date and time as the infected person. However, subsequently, if the same resource is used at the same date and time as the infected person, a search may be performed according to the present embodiment.

Note that the processes from step S101 to step S103 are an example, and the contents, the order, or the like, of the processes performed in the communication system 1 are not limited in any way within the scope of the gist of the present invention.

As described above, the user is able to use a resource by avoiding operations involving the user's physical contact with the resource terminal 5 at the start of use, at the end of use, or the like when using a predetermined reserved resource. That is, it is possible to reduce the risk of infection with a particular infectious disease that may occur by contacting the resource terminal 5, and the user is able to feel safe in using a resource.

### [Main Effects of Embodiment]

As described above, according to the first embodiment, the capturing/acquiring unit 46 of the communication terminal 4 acquires the code information embedded in the QR code by capturing the QR code displayed by the resource terminal 5 used in the resource reserved by the user (step S47), and the transmitting/receiving unit 41 transmits the user identification information of the user using the resource associated with the acquired code information, to the resource management apparatus 2 as a start request for starting use of the resource (step S62). The registering unit 28 of the resource management apparatus 2 registers the user identification information and the resource use state information in the resource state management table based on the user identification information transmitted by the communication terminal 4 (steps

S63, S64). Accordingly, the resource management apparatus 2 can manage users who use the resource in a unified manner, in accordance with the use of the resource. This facilitates the management of users in using a resource. As a result, one of the additional effects is that the user can focus on an event such as a conference held at the resource.

### Second Embodiment

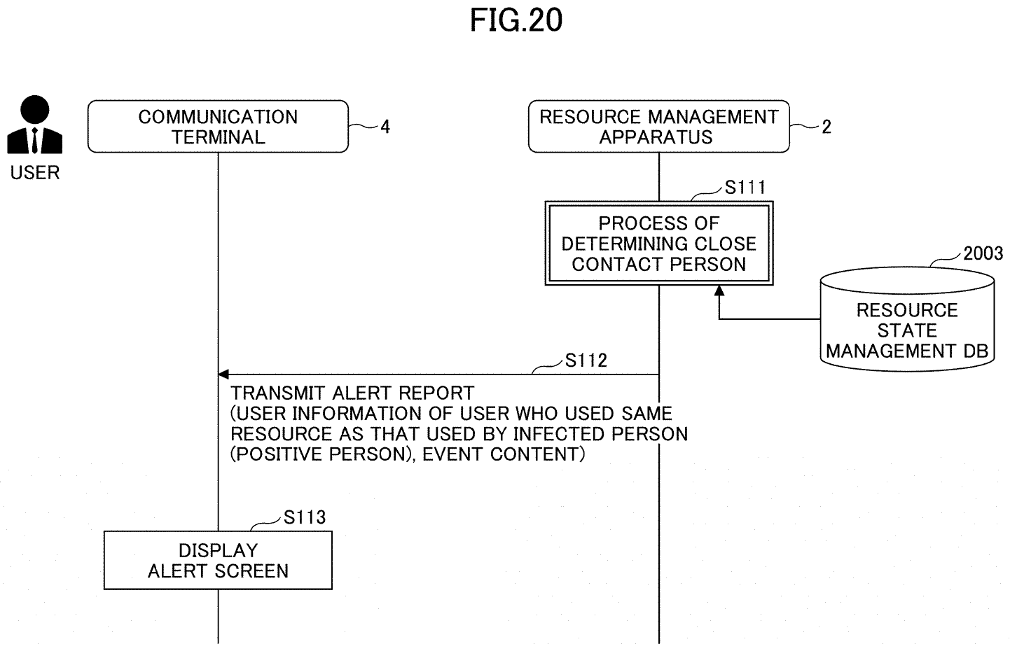

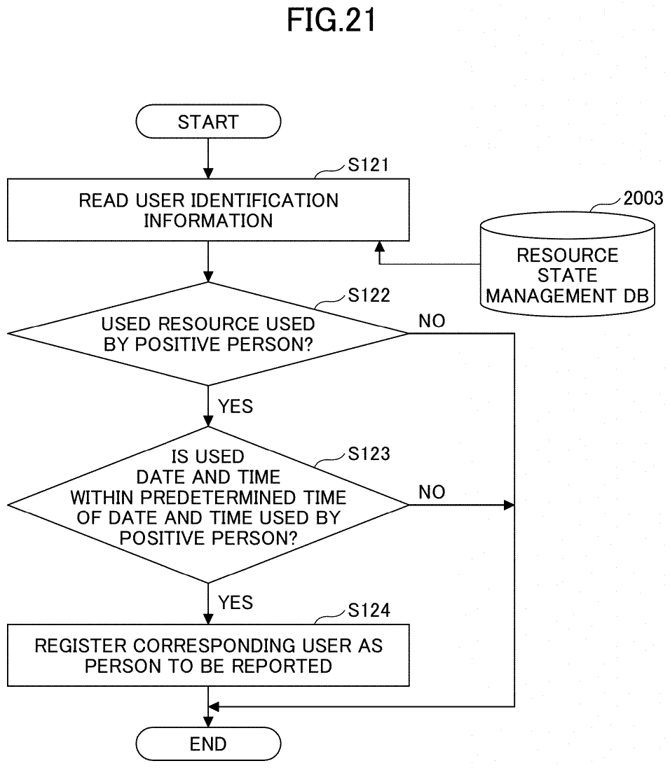

Next, a second embodiment will be described with reference to FIGS. 20 to 22. FIG. 20 is a sequence diagram illustrating an example of a process for determining a contact person and a process for sending a report to a contact person according to the second embodiment. In the second embodiment, the system configuration, hardware configuration, and each functional configuration in which the application is activated according to the user operation, the check in process with respect to the reserved resource, and the infected person registration process are performed, are the same as those of the processes up to step S93 of the first embodiment. In the second embodiment, in step S111, the searching/determining unit 25 performs the process of determining a close contact person based on the flow chart illustrated in FIG. 21.

FIG. 21 is a flowchart illustrating an example of a process for determining a contact person according to the second embodiment. First, after the registration process of the infected person performed in step S93, in step S121, the storage/reading processing unit 29 reads the resource state management table (the resource state management DB 2003, see FIG. 6) using the user identification information of the infected person as the search key.

Subsequently, in step S122, the searching/determining unit 25 determines whether the same resource as the resource used by the infected person was used by a user among the users corresponding to the user identification information that is read. At this time, if a user of the user identification information that is read has not used the same resource as the resource used by the infected person (NO in step S122), the flow is exited.

On the other hand, when the same resource as the user used by the infected person is used (YES in step S122), in step S123, the searching/determining unit 25 further determines whether the date and time used is within a predetermined time (a predetermined period) of the date and time used by the infected person (step S123). Here, if the used date and time is outside the predetermined time (the predetermined period) of the date and time of the infected person (NO in step S123), the searching/determining unit 25 regards the user of the user identification information as having no problem, and exits from the flow.

If the used date and time is within the predetermined time (the predetermined period) (YES in step S123), in step S124, the searching/determining unit 25 determines the user of the corresponding user identification information as a person to whom a report is to be sent, that is, an alert report with respect to the infected person, i.e., the user B, is to be sent to this person, and exits the flow.

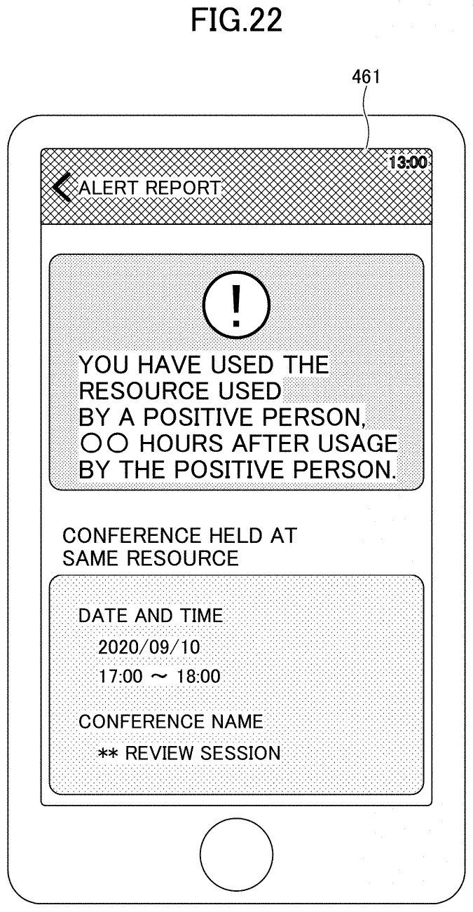

Returning to the sequence diagram of FIG. 20, subsequently, in step S112, the transmitting/receiving unit 21 transmits the alert report (an e-mail or the like) illustrated in FIG. 22 to the communication terminal 4 possessed by each of the users who is determined to be the person to whom the report is to be sent. Accordingly, the transmitting/receiving unit 41 of the communication terminal 4 receives the alert e-mail transmitted by the resource management apparatus 2. At this time, the transmitted alert e-mail includes the e-mail address and name of the user that used the resource used by the infected person (positive person), and the content of the event (date and time, conference name, or the like) held at the used resource, as user information of the user that used the same resource as that used by the infected person (positive person).

Subsequently, in step S113, the display control unit 44 displays an alert screen, for example, as illustrated in FIG. 22, based on the user information included in the received alert mail. FIG. 22 is a diagram illustrating an example of an alert screen according to the second embodiment. When the resource management apparatus 2 performs the above-described process with respect to the communication terminal 4, a user who has used the resource used by the infected person within a predetermined time (a predetermined period) can be promptly alerted. This alert is given because the risk of infection for a particular infective disease still remains through the indirect use of the resource used by the infected person, even if the infected person is not directly contacted or closely contacted.

### [Main Effects of Embodiment]

As described above, according to the second embodiment, in step S102, the searching/determining unit 25 of the resource management apparatus 2 reads the resource state management table (the resource state management DB 2003, see FIG. 6) using the user identification information of the infected person as the search key (step S121), and when the user identification information that is read indicates the usage of the same resource as the resource used by the infected person within a predetermined time (a predetermined period) of the date and time used by the infected person (YES in step S123), the transmitting/receiving unit 21 sends an alert report (e.g., an e-mail) illustrated in FIG. 22 to the communication terminal 4 possessed by the corresponding user (step S102). Accordingly, the resource management apparatus 2 can manage a user who has used the resource in a unified manner in accordance with the use of the resource. This means that, in addition to facilitating the management of users in using a resource, an alert can be received by a user if the resource used by an infected (positive) person infected with a specific infective disease, is used by the user within a specified period of time, allowing appropriate subsequent actions to be taken by the user.

### [Other Applications]

While the embodiments for carrying out the present invention have been described, the present invention is not intended to be limited in any way to such embodiments, and various modifications and substitutions may be made without departing from the spirit and scope of the invention.

The communication system 1 illustrated in the present embodiment may also be referred to as a web service. Web services are a variety of services provided through the application of Internet-related technologies. For example, web services include conference room rental services and various rental services. A system that uses web services is referred to as a use system.

Further, the communication system 1 described in the present embodiment can be further divided into a larger number of processing units depending on the processing contents. Alternatively, one processing unit can be divided to include more processes.

The functions of each of the above-described embodiments can be implemented by one or more processing circuits. Herein, a “processing circuit” includes a processor programmed to perform each function by software such as a processor implemented by electronic circuits, devices such as an Application Specific Integrated Circuit (ASIC), a digital signal processor (DSP), a field programmable gate array (FPGA), a System on a chip (SOC), a Graphics Processing Unit (GPU), a conventional circuit module, or the like, designed to perform each function as described above.

According to one embodiment of the present invention, there is an effect that the management of users in using a resource can be facilitated.

The resource management system, the resource management apparatus, and the recording medium are not limited to the specific embodiments described in the detailed description, and variations and modifications may be made without departing from the spirit and scope of the present invention.

What is claimed is:

1. A resource management system comprising:
a resource management apparatus configured to manage a use state of a predetermined resource; and
a communication terminal configured to communicate with the resource management apparatus, wherein
the communication terminal includes:
first circuitry; and
a first memory storing computer-executable instructions that cause the first circuitry to execute:
acquiring code information embedded in a predetermined code by capturing the predetermined code relating to predetermined reservation information of the predetermined resource, the predetermined code being displayed by a resource terminal that is used in the predetermined resource and configured to communicate with the resource management apparatus; and
transmitting, to the resource management apparatus, user identification information for identifying a user using the predetermined resource associated with the code information acquired at the acquiring, as a start request to start using the predetermined resource, and wherein
the resource management apparatus includes:
second circuitry; and
a second memory storing computer-executable instructions that cause the second circuitry to execute:
receiving the user identification information transmitted by the communication terminal, and
registering the user identification information and resource use state information representing the use state of the predetermined resource, based on the user identification information received at the receiving.

2. The resource management system according to claim 1, wherein
the second circuitry of the resource management apparatus is further caused to execute:
managing, in a first storage device, information including expected participant identification information of an expected participant who is scheduled to participate in a predetermined event to be held at the predetermined resource, managed in association with reservation identification information for identifying a reservation of the predetermined resource included in the code information, and wherein
the registering includes registering the user identification information and the resource use state information in the first storage device to be managed, in response to the user identification information being received at the receiving.

3. The resource management system according to claim 2, wherein
the first circuitry of the communication terminal is further caused to execute:
transmitting the reservation identification information to the resource management apparatus; and
receiving the managed expected participant identification information, transmitted by the resource management apparatus that has received the reservation identification information.

4. The resource management system according to claim 3, wherein
the first circuitry of the communication terminal is further caused to execute:
displaying, on a display, an expected participant name list of one or more names of one or more of the expected participants corresponding to the expected participant identification information received at the receiving.

5. The resource management system according to claim 4, wherein
the first circuitry of the communication terminal is further caused to execute:
accepting a selection of a user to use the predetermined resource, selected from the expected participant name list displayed on the display.

6. The resource management system according to claim 4, wherein the displaying includes displaying, on the display, a registration screen for registering infection information indicating that a particular user who has used the predetermined resource is detected to be infected by a particular infectious disease or tested positive for the particular infectious disease.

7. The resource management system according to claim 6, wherein
the displaying includes displaying, on the display, the the registration screen for registering, as the infection information, an infection detection date and time when infection or positivity with respect to the particular infectious disease is detected, and
the transmitting includes transmitting, to the resource management apparatus, particular user identification information representing the particular user, infection presence or absence information representing the infection or the positivity, and infection detection date and time information representing the infection detection date and time.

8. The resource management system according to claim 7, wherein
the second circuitry of the resource management apparatus is further caused to execute:
receiving the particular user identification information, the infection presence or absence information, and the infection detection date and time information transmitted by the communication terminal; and
managing, in a second storage device, the infection presence or absence information and the infection detection date and time information, based on the received particular user identification information, the infection presence or absence information, and the infection detection date and time information.

9. The resource management system according to claim 8, wherein
the second circuitry of the resource management apparatus is further caused to execute:
searching the user identification information managed in the first storage device, as to whether there is a contact person who has used the same resource at the same time and date as the infected particular user managed in the second storage device.

10. The resource management system according to claim 9, wherein
the second circuitry of the resource management apparatus is further caused to execute:
transmitting, to the communication terminal possessed by the contact person, contact person information relating to the contact person found at the searching, and wherein
the displaying by the communication terminal includes displaying, on the display of the communication terminal possessed by the contact person, a report that contact has been made with the infected particular user, based on the contact person information transmitted by the resource management apparatus.

11. The resource management system according to claim 9, wherein
the searching includes searching the user identification information managed in the first storage device, as to whether there is an in-predetermined time user who has used a resource used by the infected particular user within a predetermined time,
the transmitting by the resource management apparatus includes transmitting, to the communication terminal possessed by the in-predetermined time user, information reporting usage within the predetermined time, in response to finding the in-predetermined time user at the searching, and
the displaying by the communication terminal includes displaying, on the display of the communication terminal possessed by the in-predetermined time user, the information reporting the usage within the predetermined time transmitted by the resource management apparatus.

12. The resource management system according to claim 1, wherein the predetermined code is a one-dimensional code or a two-dimensional code.

13. A resource management apparatus configured to manage a use state of a predetermined resource, the resource management apparatus comprising:
circuitry; and
a memory storing computer-executable instructions that cause the circuitry to execute:
receiving user identification information transmitted by a communication terminal as a start request to start using the predetermined resource, the user identification information being used for identifying a user using the predetermined resource associated with code information embedded in a predetermined code that is acquired by the communication terminal by capturing the predetermined code displayed on a resource terminal used in the predetermined resource;
registering the user identification information and resource use state information representing the use state of the predetermined resource, based on the user identification information received at the receiving; and
transmitting, to the communication terminal, as a start response to the start request, a report that the user identification information and the resource use state information have been registered at the registering.

14. A non-transitory computer-readable recording medium storing a program that causes a computer to execute a process performed in a resource management apparatus configured to manage a use state of a predetermined resource, the process comprising:
receiving user identification information transmitted by a communication terminal as a start request to start using the predetermined resource, the user identification information being used for identifying a user using the predetermined resource associated with code information embedded in a predetermined code that is acquired by the communication terminal by capturing the predetermined code displayed on a resource terminal used in the predetermined resource;
registering the user identification information and resource use state information representing the use state of the predetermined resource, based on the user identification information received at the receiving; and
transmitting, to the communication terminal, as a start response to the start request, a report that the user identification information and the resource use state information have been registered at the registering.
